(19) United States
(12) Patent Application Publication
(10) Pub. No.: US 2025/0213751 A1
(43) Pub. Date: Jul. 3, 2025

(54) ADAPTIVE PATCHES FOR DYNAMIC ORGANS

(71) Applicant: THE UNIVERSITY OF NORTH CAROLINA AT CHAPEL HILL, Chapel Hill, NC (US)

(72) Inventors: Juliane Nguyen, Chapel Hill, NC (US); Parth Chansoria, Chapel Hill, NC (US); Tom Egan, Chapel Hill, NC (US)

(21) Appl. No.: 18/853,592

(22) PCT Filed: Apr. 3, 2023
(86) PCT No.: PCT/US2023/017288

§ 371 Date: Oct. 2, 2024

Related U.S. Application Data
(60) Provisional application No. 63/326,982, filed on Apr. 4, 2022.

(51) Int. Cl. A61L 27/52; A61L 27/22; A61L 27/36; A61L 27/44; A61L 27/54; A61L 27/56

(52) U.S. Cl. CPC A61L 27/52; A61L 27/222; A61L 27/3604; A61L 27/3679; A61L 27/3687; A61L 27/44; A61L 27/54; A61L 27/56; A61L 2300/216; A61L 2300/30; A61L 2300/412; A61L 2300/442; A61L 2300/626; A61L 2430/20; A61L 2430/22; A61L 2430/34

(57) ABSTRACT

According to some embodiments, a patch for organ and/or tissue application is provided which comprises a biocompatible polymeric material having an auxetic architecture and further matching a stiffness ratio and/or Poisson's ratio of an organ or tissue to which the patch is applied. According to some further embodiments, biocompatible patches and methods of making the same are provided comprising providing a layer of hydrogel and forming an auxetic architecture by selectively crosslinking areas of the hydrogel via exposure to light, wherein the auxetic architecture is selected to match a stiffness ratio and Poisson's ratio of an organ to which the biocompatible patch is applied.

### RELATED APPLICATION DATA

The present application claims priority pursuant to Article 8 of the Patent Cooperation Treaty to U.S. Provisional Patent Application Ser. No. 63/326,982 filed Apr. 4, 2022, which is incorporated herein by reference in its entirety.

### STATEMENT REGARDING FEDERALLY SPONSORED RESEARCH

This invention was made with government support under Grant Nos. R01CA241679, R01EB023262, and R21GM135853, awarded by the National Institutes of Health. The Government has certain rights in the invention.

### FIELD

The present invention relates to patches addressing the demanding mechanics of dynamic organs and, in particular, to patches having auxetic architectures matching mechanical and/or rheological properties of various organs.

### BACKGROUND

The last twenty years has seen a significant increase in the development and use of therapeutic patches to promote human organ repair and regeneration. These patches have served a range of applications including the treatment of myocardial infarction (MI), cancer, and chronic wound healing. However, one of the biggest challenges to developing effective patch-based therapeutics is accommodating the complex mechanics of dynamic organs (lung, heart, bladder, etc.). These organs are characterized by intrinsic anisotropy—the ability to exhibit different orientation of the cellular and extracellular matrix (ECM) components across different directions (directional anisotropy) or spatial regions/zones (spatial anisotropy). In addition to anisotropy, these dynamic organs undergo repetitive volumetric deformation, further posing design challenges for patches. For example, cardiac tissue contracts in both the longitudinal and transverse directions during systole. This imparts an auxetic surface deformation characteristic, where the surface expands or contracts across multiple directions simultaneously. Similar to the cardiac tissue, due to the interspersed smooth muscle fibers, the stomach also displays auxetic characteristics. For lung and the skin tissues containing interspersed collagen fibers, internal forces due to ingression of air (in the lung) or deformation of the underlying skeletal muscle (for the skin) impart auxetic characteristics.

While there has been tremendous progress in developing smart patches to aid tissue regeneration, most patches are isotropic or homogeneous and lack the auxetic features necessary to withstand the volumetric deformation.

### SUMMARY

The foregoing disadvantages are addressed by compositions, architectures, and methods described herein. In one aspect, a patch for organ or tissue application comprises a biocompatible polymeric material having an auxetic architecture matching a stiffness ratio and Poisson's ratio of the organ or tissue to which the patch is applied. Patches having architectures herein are not limited to any specific organ and/or tissue. In some embodiments, for example, the auxetic architecture has maximum stiffness ratio of 1 to 5 and a maximum Poisson's ratio of −1.2 to −0.05. The auxetic architecture may also be anisotropic. Moreover, the biocompatible polymeric material comprises a hydrogel, in some embodiments. Suitable hydrogel materials can include GelMA, PEGDA, gelatin norbornene, methacrylated decellularized ECM, hyaluronic acid methacrylate or combinations there. The hydrogel may be a single layer or multiple layers. In some embodiments, layers of the hydrogel have different compositions. For example, a layer of hydrogel interfacing with surfaces of the organ or tissue can exhibit different composition and/or surface functionalities relative to an opposing exterior hydrogel layer. Surfaces of the patch can be populated with moieties operable to form hydrogen bonds and/or ionic bonds with organ/tissue surfaces. In some embodiments, the moieties include carboxyl and/or hydroxyl groups. For example, the hydrogel, such as gelatin methacroyl, can be modified with acrylic acid. In some embodiments, the hydrogel can have a degree of functionalization of 0.1-100%. Functionalization with carboxyl groups, in some embodiments, can range from 1-50% v/v or 0.01 to 100 molar equivalent relative to methacrylate groups of the hydrogel. Moreover, nobornene functionalized hydrogel matrices can include thiolated crosslinkers, such as PEG, thiolated gelatin, or dithiothreitol (DTT). Crosslinkers, for example, can be present at 0.01 to 2 molar equivalent of the norbornene groups.

Further, the hydrogel architecture can comprise cationic metals for forming ionic bonds between patch and tissue surfaces. Suitable cationic metals can include alkali metals and alkaline earth metals. Cationic metals can be present in the hydrogel at a concentration of 0.001M to 1M, in some embodiments. As described further herein, the hydrogel architecture may also include a UV absorber component and photoinitiator.

As described further herein and in the accompanying Appendix, the auxetic architecture comprises apertures of repeating shape or patterned apertures. In comprising apertures, the auxetic architecture can employ lattice structures or lattice-like structures. The apertures can have any desired shape consistent with the technical objectives described herein. Aperture shape can be chosen in conjunction with identity of the hydrogel material to provide the desired stiffness ratio and Poisson's ratio. In this way, the auxetic architecture can be tailored to specific properties of the organ to which the patch is applied. In some embodiments, the apertures have a shape selected from the group consisting of re-entrant honeycomb, chiral truss, lozenge truss, orthogonal oval voids, arrowheads, pinwheels, sinusoidal ligaments, and orthogonal truss.

Additionally, in some embodiments, apertures of the patch are filled with material blocking or inhibiting the passage of liquid and/or gas. In such embodiments, the patch can be used to seal and treat punctures or surgical openings in organs, including but not limited to, the lungs, stomach, intestine, bladder, heart, kidneys, uterus, ureter, blood vessels, skin, and colon. Young's modulus of the filler material, in some embodiments, is less than that of the biocompatible polymeric material.

Apertures of the patch may also be filled with one or more therapeutic agents, including pharmaceuticals, biologics, nucleic acids, proteins, or cellular species. Therapeutics agents can be selected according to the desired biological response to be elicited. In some embodiments, a therapeutic agent is operable for wound healing. Therapeutic agents may be directly applied to the biocompatible polymer or hydrogel forming the apertures. Alternatively, therapeutic agents can be associated with a biocompatible carrier. The carrier, in some embodiments, comprise exosomes.

In another aspect, methods of healing organs or tissues are described herein. In some embodiments, a method comprises applying a patch to a damaged area of the organ or tissue, the patch comprising a biocompatible polymeric material having an auxetic architecture matching a stiffness ratio and Poisson's ratio of the organ or tissue. The patch can have any of the composition, architecture, and/or properties described hereinabove. In some embodiments, for example, apertures of the patch are filled with material blocking or inhibiting the passage of liquid and/or gas. In such embodiments, the patch can be used to seal and treat punctures or surgical openings in organs and/or tissues, including but not limited to, the lungs, stomach, intestine, bladder, heart, kidneys, uterus, ureter, blood vessels, skin, and colon. The patch may also contain one or more therapeutic agents to promote healing.

In another aspect, methods of making biocompatible patches are described herein. A method comprises providing a layer of hydrogel, and forming an auxetic architecture by selectively crosslinking areas of the hydrogel via exposure to light, wherein the auxetic architecture is selected to match a stiffness ratio and Poisson's ratio of an organ or tissue to which the biocompatible patch is applied. Organs and tissues can include the lungs, skin, stomach, heart, bladder, intestine, kidneys, uterus, ureter, blood vessels, and colon. Crosslinking selected areas of the hydrogel to provide the auxetic architecture can be administered by any technique(s) consistent with the technical objectives described herein. In some embodiments, digital light projection is employed to achieve the selective crosslinking. In other embodiments, three dimensional printing techniques are employed, including selective deposition of hydrogel on a build platform according to parameters set forth in an electronic format of the patch design. The deposited hydrogel is then crosslinked or cured. The patch can have any composition, architecture, and/or properties described hereinabove. In some embodiments, the hydrogel composition may further comprise one or more UV absorbers. For example, the hydrogel composition may comprise curcumin, such as curcumin nanoparticles. Additional UV absorbers can include FD&C yellow food dye, GelOrange or Sunset Yellow dyes. UV absorber can be present in the hydrogel in an amount of 0.0001 mg/ml to 10 mg/ml, in some embodiments. Use of an UV absorber can enhance resolution of various structural features of the patch. In some embodiments, UV absorber can enable the production of patch structural features having a resolution of 50-500 μm.

In some embodiments, a method further comprises filling apertures of the patch with material blocking or inhibiting passage of liquid and/or gas. Moreover, a method can further comprise filling the apertures with one or more therapeutic agents. The one or more therapeutic agents can comprise a pharmaceutical, biologic, nucleic acid, protein, or cellular species. The therapeutics agent may be directly applied to the biocompatible polymer or hydrogel forming the apertures. Alternatively, the therapeutic agent(s) can be associated with a biocompatible carrier. The carrier, in some embodiments, comprise exosomes.

These and other embodiments, objects, advantages, and features of the technology are further illustrated and set forth in the following non-limiting examples of the detailed description.

### BRIEF DESCRIPTION OF THE FIGURES

Aspects of the technology presented herein are described in detail below with reference to the accompanying drawing figures, wherein:

FIG. 1A illustrates PAAx patches can stretch in multiple directions simultaneously (auxetic property), and the degree of stretchability in different directions can also be different (anisotropy), in accordance with some embodiments of the present technology;

FIG. 1B illustrates PAAx patch fabrication, in accordance with some embodiments of the present technology;

FIG. 1C illustrates differential attachment of cells (3T3 fibroblasts) in the GelMA and PEGDA layers, in accordance with some embodiments of the present technology;

FIG. 1D shows cells cultured in the presence of the patches demonstrate 100% cell viability over 5 days, and no difference in cellular metabolic activity (MTS assay) is observed compared to controls (no patch), in accordance with some embodiments of the present technology;

FIG. 2A shows different patch architectures which demonstrate auxetic mechanical characteristics, in accordance with some embodiments of the present technology;

FIG. 2B shows patches incubated in PBS for 3h demonstrate swelling, leading to an increase in surface area, in accordance with some embodiments of the present technology;

FIG. 2C illustrates bulk material properties of the constitutive materials derived via mechanical testing of dog-bone shaped patches, in accordance with some embodiments of the present technology;

FIG. 2D shows example outputs of the computational models of the patches, which demonstrate that uniaxial stretching of the patches also leads to stretching in the lateral direction, in accordance with some embodiments of the present technology;

FIG. 2E illustrates functional mapping of the mechanical properties of iterations of different patch architectures, in accordance with some embodiments of the present technology;

FIG. 3A shows Ashby plots demonstrating the different stiffnesses and Poisson's ratios of organs, and patch architectures that match to the different organ ranges, in accordance with some embodiments of the present technology;

FIG. 3B illustrates selected optimized patch architectures after matching to the transverse stiffnesses of different organs, in accordance with some embodiments of the present technology,

FIG. 3C illustrates fabricated architectures for the selected patches mimicking the stiffness ratios, Poisson's ratios and transverse stiffnesses of different organs, in accordance with some embodiments of the present technology;

FIG. 3D shows tensile testing of selected patch architectures demonstrates close correlations between experimental outcomes and computational estimates in terms of transverse stiffness, stiffness ratio, and yield strain, establishing the validity of the computational models, in accordance with some embodiments of the present technology;

FIG. 3E shows comparison of the surface deformation of different organs, in accordance with some embodiments of the present technology;

FIG. 3F illustrates a balloon model mimicking bladder deformation, where the Re-en itr5 architecture was evaluated; in accordance with some embodiments of the present technology;

FIG. 3G illustrates a setup for a balloon model, which allows a constant output of air (Fₒᵤₜ) while the input (Fᵢₙ) is actuated in cycles, causing the balloon to dilate and contract, thereby stretching a patch, in accordance with some embodiments of the present technology;

FIG. 4A illustrates the mechanical properties of patches for scaling factors s=0.75, 1 and 1.5 corresponding to 15, 20, and 30 mm length, respectively, in accordance with some embodiments of the present technology;

FIG. 4B shows computational predictions of mechanical straining of different patch sizes suggest unchanged stiffness and Poisson's ratios, in accordance with some embodiments of the present technology;

FIG. 4C illustrates that transverse stiffness is inversely proportional to the square of the scaling factor, in accordance with some embodiments of the present technology;

FIG. 4D illustrates that yield strain is proportional to a scaling factor, in accordance with some embodiments of the present technology;

FIG. 4E shows expanded lung-mimicking Loz itr5 patches under different scaling factors, in accordance with some embodiments of the present technology;

FIG. 4F illustrates patch dimensions after expansion due to swelling, showing that the final patch length after swelling is proportional to the scaling factor(s) and the patch area is proportional to s2, in accordance with some embodiments of the present technology;

FIG. 4G shows experimental validation of transverse stiffness, in accordance with some embodiments of the present technology;

FIG. 4H shows experimental validation of maximum stiffness ratio, in accordance with some embodiments of the present technology;

FIG. 4I shows experimental validation of yield strain, in accordance with some embodiments of the present technology;

FIG. 5A shows a scheme of adding a thin coat of fibrin on patches to impart bioadhesiveness, in accordance with some embodiments of the present technology;

FIG. 5B shows Thrombin applied to the patches after placement on the organs for strong bioadhesion, in accordance with some embodiments of the present technology;

FIG. 5C shows a comparison of the compliance of non-auxetic patches with hexagonal holes (placed on the left lobe) and the lung-mimicking auxetic patches (Loz itr5 architecture, placed on the right lobe), in accordance with some embodiments of the present technology;

FIG. 5D shows analysis of the area covered by the patches under different ventilation cycles and demonstrates that auxetic patches conform to the lung dynamics better than the non-auxetic patches, in accordance with some embodiments of the present technology;

FIG. 5E illustrates administration of patches over the left lobe of porcine lungs (inset) and demonstrates patch conformation to the different ventilation cycles of the lung, in accordance with some embodiments of the present technology;

FIG. 5F shows analysis of the size of the patches demonstrates better conformation of the auxetic patches to the lung dynamics compared to non-auxetic patches, in accordance with some embodiments of the present technology;

FIG. 5G shows open chest surgery on rats to expose the lungs, with patches applied using a thin layer of fibrin glue, in accordance with some embodiments of the present technology;

FIG. 5H illustrates that after administration over the organ, the auxetic patches demonstrated similar deformation to the native tissue during different inhalation-exhalation cycles of the lung, in accordance with some embodiments of the present technology;

FIG. 6A illustrates hole filling PAAx patches comprising a structural lattice composed of GelMA-PEGDA bilayers and a hole-filling element of fibrin glue, in accordance with some embodiments of the present technology;

FIG. 6B illustrates patches with hole-filling fibrin and demonstrating similar expansion to patches without any hole-filling fibrin, in accordance with some embodiments of the present technology;

FIG. 6C illustrates a scheme of application of hole filling PAAx patches, in accordance with some embodiments of the present technology;

FIG. 6D illustrates variation in tidal pressure of healthy or injured lungs under physiological ventilation (PV, 7.5 ml/kg ventilation volume) and hyperventilation (HV, 12.5 ml/kg ventilation volume), in accordance with some embodiments of the present technology;

FIG. 7A illustrates PAxS patches fabricated via a facile single-layer projection-printing of a photoink (GelMA+ACA) contained in a PDMS-coated substrate, in accordance with some embodiments of the present technology;

FIG. 7B shows the optimization of CNP content within PAxS patches for high-resolution printing, in accordance with some embodiments of the present technology;

FIG. 7C illustrates patch materials demonstrate excellent biocompatibility with or without CNP as tested with viability of 3T3 cells after 48 h in culture, in accordance with some embodiments of the present technology;

FIG. 8A is a schematic showing that the combination of hydrogen, covalent, and ionic bonding impart instantaneous and strong bioadhesion and ultra-elasticity to PAxS patches, in accordance with some embodiments of the present technology;

FIG. 8B shows patches attached to a rat liver demonstrate instantaneous adhesion in <1 s sufficient to support the weight of the rat liver, in accordance with some embodiments of the present technology;

FIG. 8C shows patches demonstrating strong bioadhesion to mouse liver, in accordance with some embodiments of the present technology;

FIG. 9A illustrates different dog bone-shaped patch formulations that first underwent tensile testing to optimize the patch formulation and mechanical properties for computational modeling, in accordance with some embodiments of the present technology;

FIG. 9B shows selected auxetic and non-auxetic architectures used within computational modeling, in accordance with some embodiments of the present technology;

FIG. 9C shows computational outputs depicting internal stresses within the selected iterations of the geometries of auxetic (negative Poisson's ratio) and non-auxetic (positive Poisson's ratio) architectures using the 80% GelMA-20% ACA formulation, in accordance with some embodiments of the present technology;

FIG. 9D shows plots of patch stiffness ratios, Poisson's ratios, and yield strains and overlap with the properties of individual organs, in accordance with some embodiments of the present technology;

FIG. 9E illustrates that yield strain is always greater than that of the normal physiological strain of the organ, in accordance with some embodiments of the present technology;

FIG. 10A shows printed patches based on selected patch designs for different dynamic organs, in accordance with some embodiments of the present technology;

FIG. 10B illustrates a demonstration of patch ultra-elasticity, in accordance with some embodiments of the present technology;

FIG. 10C shows tensile testing of selected patches for different organs demonstrating a close correlation between the computational and experimental outcomes of the elastic modulus (stiffness) and the yield strains of the patches, in accordance with some embodiments of the present technology;

FIG. 10D illustrates volumetric fold change of different dynamic organs, in accordance with some embodiments of the present technology;

FIG. 10E shows Re-en itr 1 patches tested on a dilating balloon model demonstrating adherence to the balloon mechanics and a similar surface expansion to that of the balloon, in accordance with some embodiments of the present technology;

FIG. 11A shows ex vivo implantation of patches (15) over rat lungs, and comparison of a patch with square holes vs an auxetic patch (Loz itr5), in accordance with some embodiments of the present technology;

FIG. 11B illustrates ex vivo implantation of the patches (30×30 mm²) over the right lobe of porcine lungs, where auxetic patches (Loz itr4) better conformed to the lung mechanics compared with non-auxetic (honeycomb) patches, in accordance with some embodiments of the present technology;

FIG. 11C illustrates a concept demonstration of the use of minimally-invasive surgical tools to facilitate PAXS patch placement, in accordance with some embodiments of the present technology;

FIG. 11D illustrates patches conforming to a heart beat over several systolic and diastolic cycles, in accordance with some embodiments of the present technology;

FIG. 12A illustrates that there is high variability in patch deformation under different physiological motions of skin, and taking the sole and dorsum as example regions, the Poisson's ratios of various regions during dorsiflexion or plantarflexion are indicated, in accordance with some embodiments of the present technology;

FIG. 12B illustrates patch deformation relevant to the different subregions of the sole or dorsum in an anatomical foot model under dorsiflexion or plantarflexion, in accordance with some embodiments of the present technology;

FIG. 13A illustrates that anti-inflammatory, antimicrobial, and antioxidant properties of CNPs can be combined with pro-proliferative and anti-inflammatory properties of MSC-derived exosomes (MSC-Exos) to improve wound healing, in accordance with some embodiments of the present technology;

FIG. 13B illustrates that due to their intrinsic bioadhesiveness, PAxS patches allow the attachment of fluorescently-labeled exosomes to their surface, in accordance with some embodiments of the present technology;

FIG. 13C shows in vitro scratch assays with 3T3 fibroblasts demonstrating that patches laden with both CNPs and MSC-Exos clearly outperform patches laden with CNPs and the control groups (no patches), in accordance with some embodiments of the present technology;

FIG. 13D shows PAxS patches laden with CNPs and MSC-Exos demonstrated significantly greater wound closure compared with other treatment groups after a week of administration over cutaneous wounds in mice, in accordance with some embodiments of the present technology;

FIG. 14A shows VF-PAxS patches fabricated by filling the voids of PAXS patches using a reduced concentration of matrix containing GelMA and ACA, in accordance with some embodiments of the present technology;

FIG. 14B shows PAxS patches synthesized using a 20% lower concentration of material than that used for the lattices (80% v/v GelMA and 20% v/v ACA solutions) demonstrating auxetic properties on balloon deformation without opening matrix pores, in accordance with some embodiments of the present technology;

FIG. 14C illustrates the concept of using VF-PAxS patches for the treatment of pulmonary air leakage in an in vivo rat model, in accordance with some embodiments of the present technology;

FIG. 14D shows a lung-mimicking VF-PAxS patch and its placement over rat lung, in accordance with some embodiments of the present technology; and

FIG. 14E illustrates that after inducing pulmonary injury, the tidal ventilation pressure during dilation of the lung reduced from 7.2 to 5.8 mmH₂O and VF-PAxS patches fully restored the ventilation pressure to physiological levels, while simple PAxS patches were unable to improve the lung function due to air leak through the empty voids within the patch lattices, in accordance with some embodiments of the present technology.

### DETAILED DESCRIPTION

Embodiments described herein can be understood more readily by reference to the following detailed description and examples and their previous and following descriptions. Elements, apparatus, and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less (e.g., 1.0 to 5.3, or 4.7 to 10.0, or 3.6 to 7.9).

All ranges disclosed herein are also to be considered to include the end points of the range, unless expressly stated otherwise. For example, a range of “between 5 and 10” or “5 to 10” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

Additionally, in any disclosed embodiment, the terms “substantially,” “approximately,” and “about” may be substituted with “within [a percentage] of” what is specified, where the percentage includes 0.1, 1, 5, and 10 percent.

### Example 1—Auxetic Patches for Dynamic Organs

According to some embodiments of the present technology, anisotropic and auxetic patches, for example hydrogel patches, are provided for adaptation to dynamic organs. As will be appreciated, current hydrogel or fabric patches for organ repair are generally not designed to conform to the complex mechanics of dynamic organs such as the lung or heart. According to aspects of the present technology, improved patches are provided which can comprise a biocompatible and bilayered, hydrogel-based patch platform, consisting of a non-fouling top layer and a cell adhesive bottom layer, that caters to the anisotropic and auxetic characteristics of dynamic organs. Integrated computational and experimental studies were used to screen over 116 unique anisotropic-auxetic architectures to establish design rules and tailor the patches to a broad range of target organ dynamics. The patches were then validated in ex vivo and in vivo animal models, where the auxetic patches outperformed non-auxetic patches in conforming to the volumetric dilation-contraction of dynamic organs. Next, preclinical design and testing of novel hole-filling auxetic patches was performed. These patches composited with fibrin robustly reduced pulmonary air leakage in live anesthetized rats with surgically induced lung puncture. This is the first demonstration of a rational patch design framework that features both anisotropic and auxetic properties to cater to a wide range of organ dynamics. These studies can further form a basis for future clinical development of biomimetic patches.

Auxetic structures are characterized by lattice elements, that feature intermediate voids that can enlarge when the lattice is deformed. As a result, these architectures expand across multiple directions simultaneously, thereby demonstrating a negative Poisson's ratio. While the dynamic organs tend to expand across multiple directions simultaneously (auxetic properties), in most cases, the amount of expansion is also different across different directions (anisotropy). In such a case, a combination of auxetic characteristics and anisotropy can allow the patches to mimic the mechanics of the different dynamic organs. Within the auxetic architectures, anisotropic characteristics can be imparted by changing the designs of lattices across different directions which can allow biomimicry of organ mechanics. Some examples of anisotropic and auxetic patches include the creation of auxetic re-entrant honeycomb architectures or auxetic sinusoidal ligament architectures to mimic the cardiac tissue anisotropy and stiffness. Despite progress in the development of complex patches, the majority of studies have mainly focused on the treatment of cardiovascular diseases, and while cardiovascular diseases incur a tremendous socioeconomic and healthcare impact, there is also a need to tailor anisotropic and auxetic patches for other pathologies.

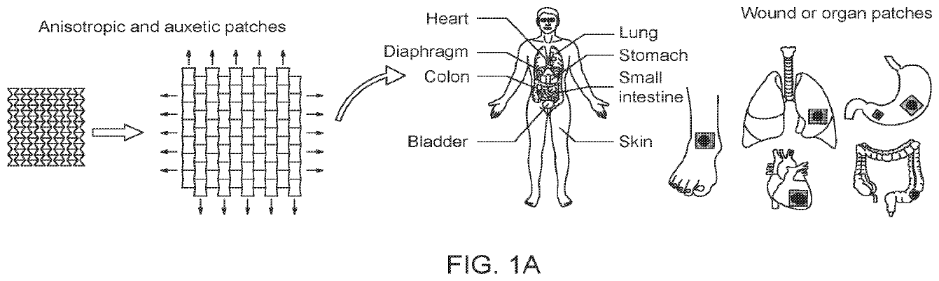

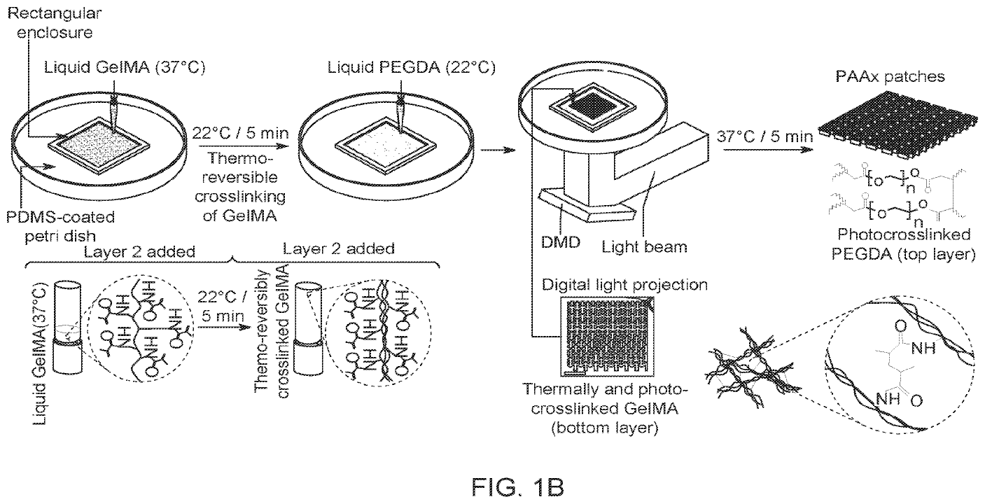

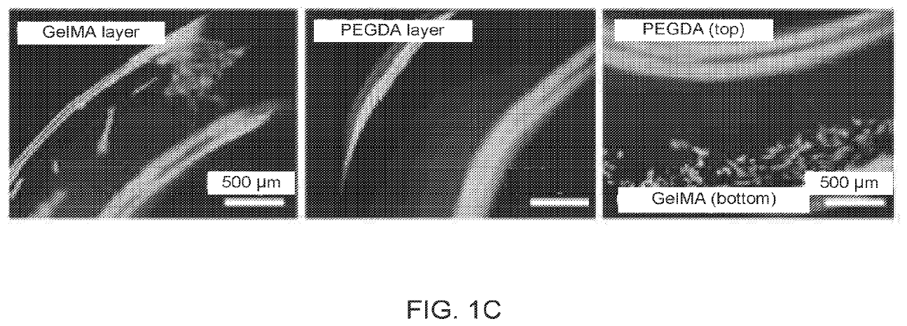

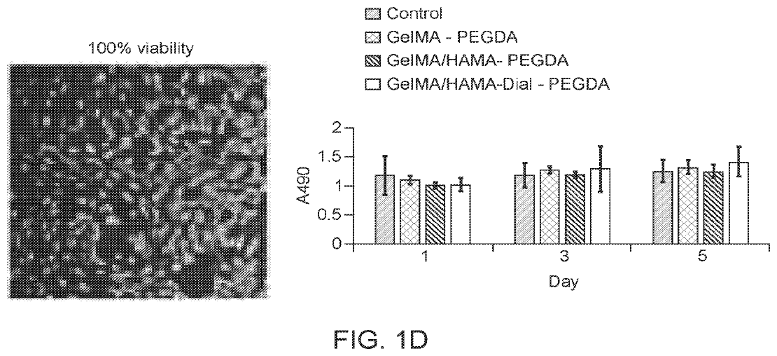

According to aspects of the present technology, a rational design framework and guidelines is provided for the development of adaptive anisotropic and auxetic patches to mimic the mechanics of dynamic organs including the lung, heart, stomach, bladder, intestines, and the skin, as can be seen for example in FIG. 1A. With reference to FIGS. 1A-1D, projection-printed anisotropic and auxetic PAAx patches and their properties and use cases are presented. By screening over 116 patches and exploiting in silico modeling a versatile platform of patch architectures that can be applied to a broad range of dynamic organs is designed. Further, it is demonstrated demonstrate how these optimized patches conform to the organ mechanics over a bladder-mimicking balloon expansion-contraction model, ex vivo rat and pig lungs, and the lungs in live anesthetized rats. Additionally, novel hole-filling auxetic patches for the treatment of lung puncture in a rat model is developed. As seen in FIG. 1A, PAAx patches can stretch in multiple directions simultaneously (auxetic property), and the degree of stretchability in different directions can also be different (anisotropy). Potential applications include treatment of organ pathologies such as chronic wound healing, pneumothorax, and cardiac diseases.

PAAx patch composition and fabrication. Patches that combine active and passive layers have enhanced clinical translation potential compared to single-material patches. In two-layer patches, the active side makes contact with the diseased organ, while the passive side prevents interactions with external environmental components to further prevent infection or scar formation. A digital light projection was used to fabricate a bilayered hydrogel patch system consisting of a non-adhesive polyethylene glycol-diacrylate (PEGDA) top layer and a cell-adhesive bottom layer consisting of gelatin methacryloyl (GelMA). The fabrication scheme of the projection-printed anisotropic and auxetic (PAAx) patches is shown in FIG. 1B. As shown in FIG. 1B, for PAAx patch fabrication, first layer of liquid GelMA (37° C.) is added to a rectangular enclosure within a PDMS coated petri dish. After thermo-reversible crosslinking of GelMA at 24° C. for 5 min, a second layer of PEGDA at 24° C. is added. The patches are then projection-printed to produce selective intra- and inter-layer photocrosslinking of the GelMA and PEGDA layers. The enclosure is then incubated at 37° C. to remove the non-photocrosslinked GelMA and derive the bilayered patches. This rapid fabrication method allowed patch fabrication in under two minutes. The top PEGDA layer insulates the patch from the interstitial fluid and prevents undesired protein adsorption or cell attachment, while the bottom GelMA layer, that makes contact with the tissue, allows active interactions with host cells and ECM. To clearly demarcate the GelMA and PEGDA layers, the thermo-reversible crosslinking property of GelMA can be exploited to crosslink the bottom layer at room temperature (24° C.) prior to adding liquid PEGDA (also at 24° C.) on top. After photo-projection printing and heating to 37° C., the non-photocrosslinked GelMA dissolved away to yield a bilayered patch, with distinct PEGDA and GelMA layers, constituting the anisotropic-auxetic architecture that can conform to the mechanics of a specific dynamic organ.

Favorable biocompatibility with the host is a necessary first step towards clinical translation of any biomedical product. Accordingly, patch cytocompatibility was assessed. FIG. 1C demonstrates the selective attachment of 3T3 fibroblasts across the different layers (both top and side views) of the PAAx patches, more particularly showing differential attachment of cells (3T3 fibroblasts) in the GelMA and PEGDA layers. The GelMA layer allows cell adhesion and proliferation, while the top PEGDA layer is a non-fouling layer without any cell attachment. The side view also demonstrates clear distinction in cell attachment only to the bottom GelMA layer. For each patch, the cells only attached to the GelMA layer and not the PEGDA layer. Therefore, the PEGDA layer can act as a non-fouling layer that isolates the top of the patch from undesired adhesion of proteins present in the interstitial fluid. The 3T3 fibroblasts also proliferated in the presence of the patches, demonstrating 100% viability and no decrease in metabolic activity (FIG. 1D) compared to controls. As seen in FIG. 1D, cells cultured in the presence of the patches demonstrate 100% cell viability over 5 days, and no difference in cellular metabolic activity (MTS assay) is observed compared to controls (no patch) (p>0.05, group-wise comparisons carried out via Tukey's honestly significant difference (HSD) post-hoc tests).

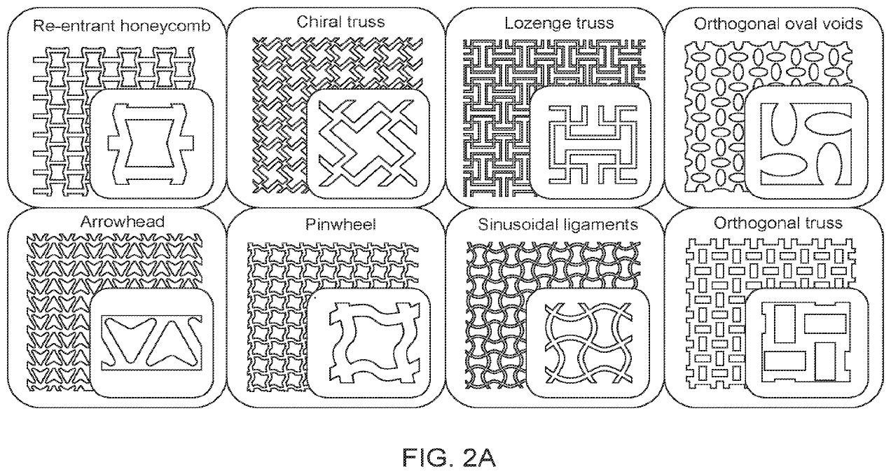

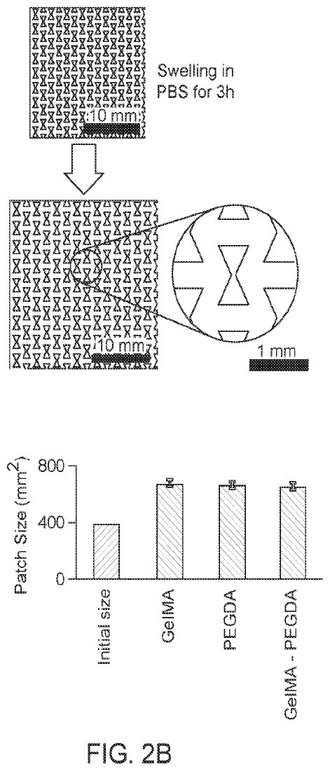

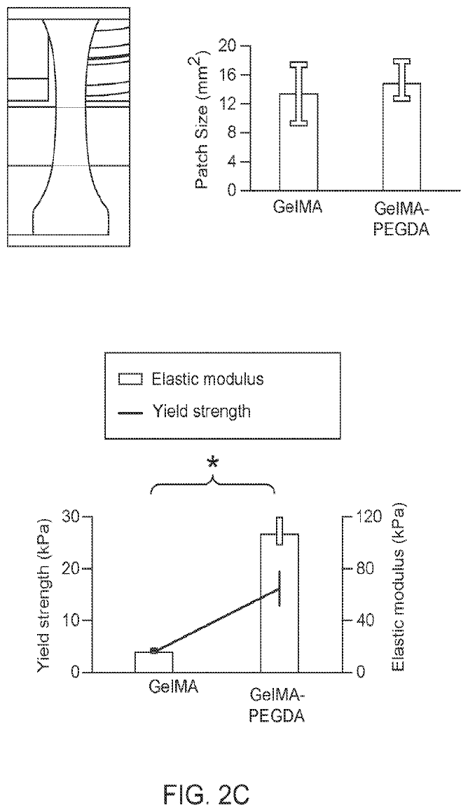

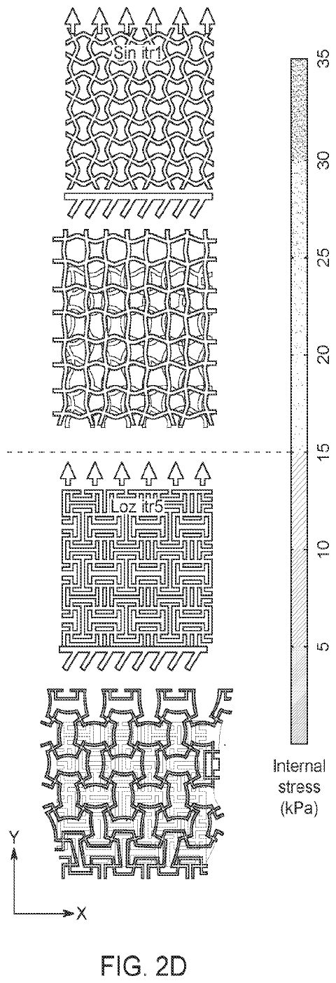

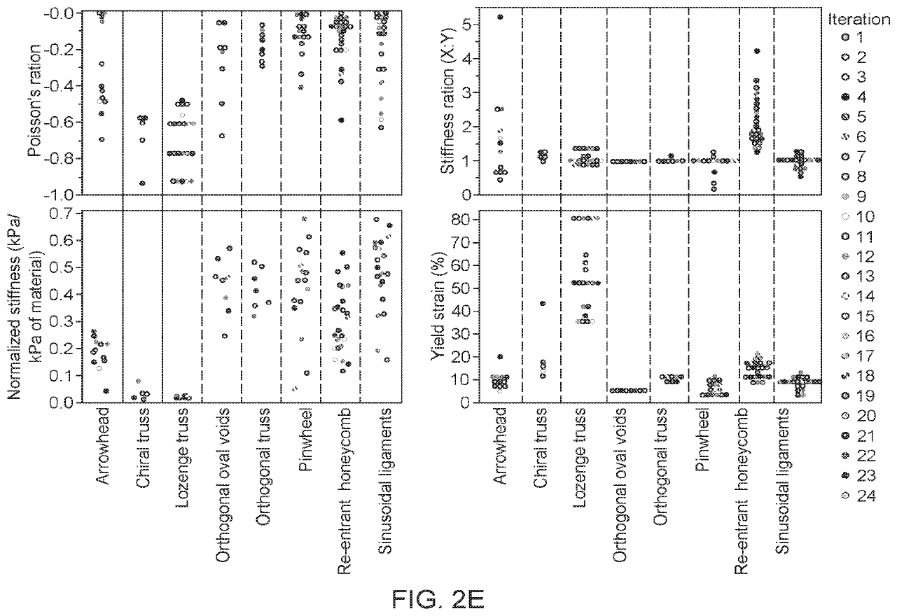

Rationally designed PAAx patches to mimic dynamic organ mechanics. The auxetic surface deformation of dynamic organs demands patch architectures with a negative Poisson's ratio, while the anisotropic cell and ECM distribution demands different patch compliance in different directions. While there is a wide selection of design choices for auxetic architectures, few patches mimic the native organ's Poisson's ratio and its anisotropic stiffness. To accomplish this, over 116 patch designs were screened based on different iterations of the design parameters such as the width (w), height (h), radius (r), and thickness (t), etc. of eight different auxetic architectures, illustrated in FIG. 2A. As a general design criterion, each patch was kept constant in size to conform to a 20×20 mm² shape. The structural mechanics of the patches were computationally modeled and experimentally verified. This approach allows the development of a versatile auxetic patch platform that can be appropriately tailored to different pathologies in different organs.

To derive the geometry and material properties for the computational models, the swelling and the mechanical properties of the bulk materials was investigated. A 20 mm patch expanded to up to 26 mm (FIG. 2B, p<0.05, posthoc analysis via Tukey's HSD) irrespective of the bi-layered material combination (p>0.05, posthoc comparisons via Tukey's HSD). As a result, the computational model was calibrated to scale-up the geometry to 26 mm (isotropic scaling factor of 1.3). To determine the bulk material properties, dog-bone shaped bi-layered patches were created for tensile testing (FIG. 2C), a commonly studied geometry. The average Young's modulus (stiffness) of the materials was ˜100 kPa and the average yield strength was ˜17 kPa, greater than pure GelMA patches (4.1 kPa average Young's modulus and 5 kPa average yield strength). As shown in FIG. 2C bulk material properties of the constitutive materials derived via mechanical testing of dog-bone shaped patches. These results were used to define the material properties in the computational models to derive the mechanical properties of the PAAx patches. * p<0.05 (posthoc analysis via Tukey's HSD).

FIG. 2D demonstrates the deformation of some selected architectures under external load. Upon patch expansion in one direction, these architectures also underwent expansion in the lateral direction, a typical auxetic behavior. Examination of the different design iterations of the architectures as can be seen in FIG. 2E shows that the chiral and lozenge truss had the most negative Poisson's ratios (−0.6 to −0.95, which means that the patch dimension increases by 60% to 90% in the transverse direction, when stretched by 100% in the longitudinal direction) making them highly suitable for organs such as the lung which undergo almost identical deformation in the transverse and the longitudinal directions (Poisson's ratios close to −1). Such a highly negative Poisson's ratio can be attributed to the configuration of the lattice elements (repeating units) of the chiral and lozenge truss architectures. The elements of these architectures are configured such that, upon stretching, the voids expand almost equally across both longitudinal and transverse directions. Other architectures, due to the differences in the amount of expansion of voids in different directions upon stretching, had Poisson's ratios between 0 to −0.6.

Stiffness ratios of different organs subsequently investigated and characterized since a patch that is softer than the organ or matches the stiffness in one direction can easily conform to organ dynamics in different directions. Importantly, the re-entrant honeycomb and arrowhead architectures demonstrated the largest range of longitudinal: transverse stiffness ratios since the design of the lattice elements is directionally asymmetrical and thus behaves differently in different directions within the patches. Furthermore, changing the height and width further affected the stiffness ratios of these architectures. In contrast, architectures and their iterations that are symmetrical in nature in different directions had stiffness ratios <1.5, which means that the stiffness in the transverse direction was almost identical to that in the longitudinal direction. These include mostly the chiral lozenge truss architectures, and some iterations of pinwheel, orthogonal oval voids and orthogonal truss architectures. The normalized stiffness was also characterized, which is the ratio of the stiffness of the patch to the stiffness of the patch material. The chiral and lozenge truss architectures exhibited the lowest normalized stiffness (soft patches, <0.1 kPa/1 kPa of material) since these patches exhibited the highest mechanical compliance through increased foldability of the lattice elements. Foldability is directly related to the amount of expansion the voids can undergo during stretching. The voids, between the lattice elements of Chiral and Lozenge truss architectures, increase substantially across all directions when these are stretched. Therefore, for a material with a 100 kPa elastic modulus, these patches exhibited a stiffness of ≤10 kPa or less. The arrowhead architecture can also be categorized as a soft patch with a normalized stiffness of 0.3 kPa/1 kPa (i.e., the patch will have a stiffness up to 30 kPa when the material is 100 kPa), while other patches had normalized stiffnesses up to 0.7 kPa/1 kPa of material (i.e., the patch will have a stiffness up to 70 kPa when the material is 100 kPa).

Yield strain is another important parameter to ensure that the patches do not break or lose their elasticity under normal physiological functioning. Due to the unique packing of the lattice elements which improves stretchability, the chiral and lozenge truss architectures had the highest yield strain, stretching up 80% of their original length without losing elasticity. In contrast, other patches had yield strains ≤30%.

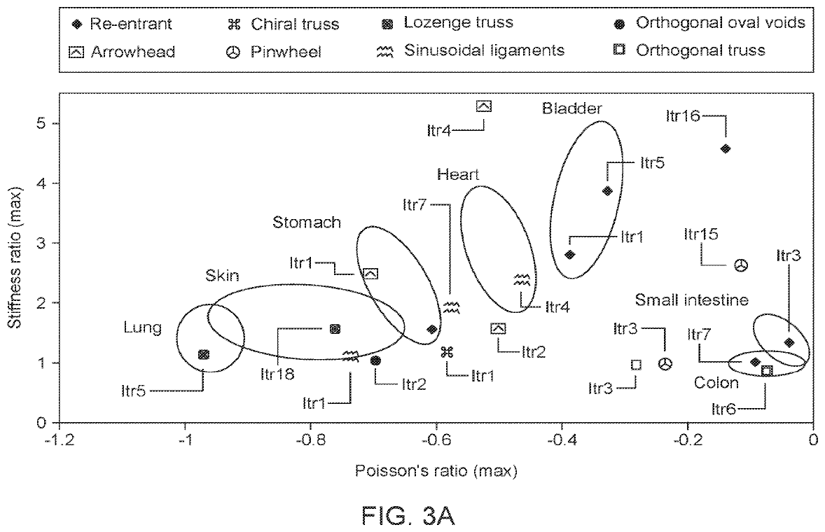

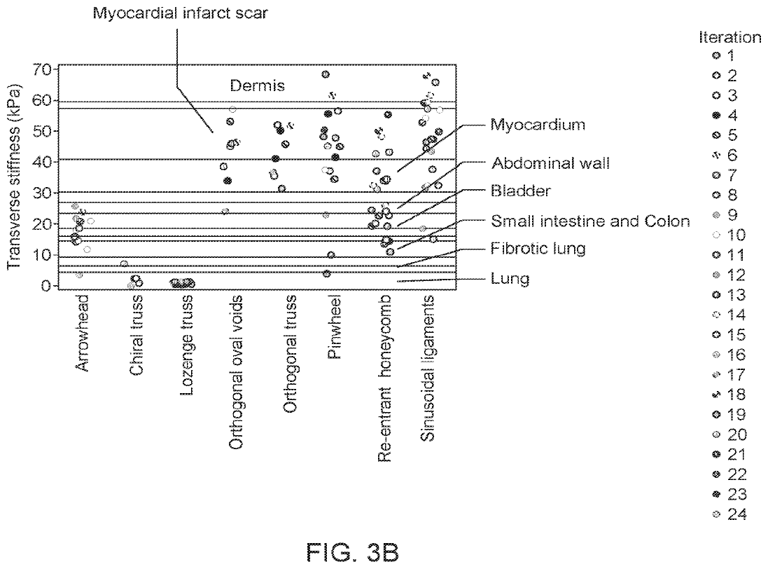

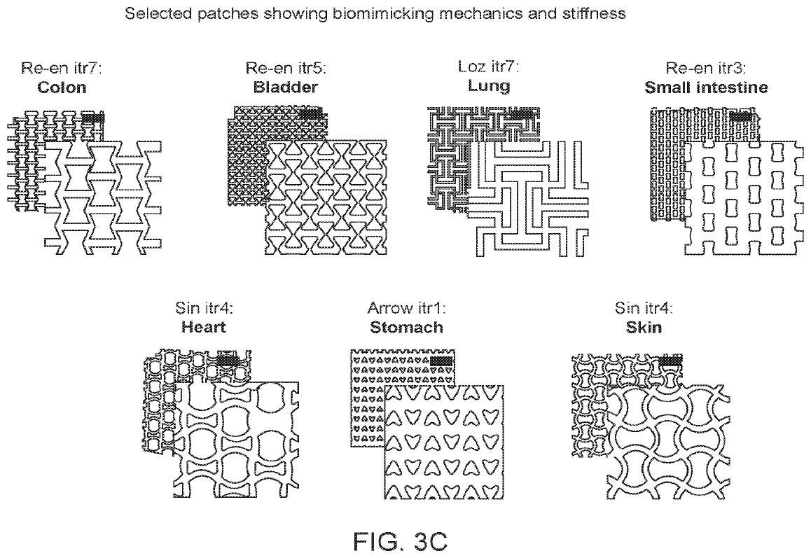

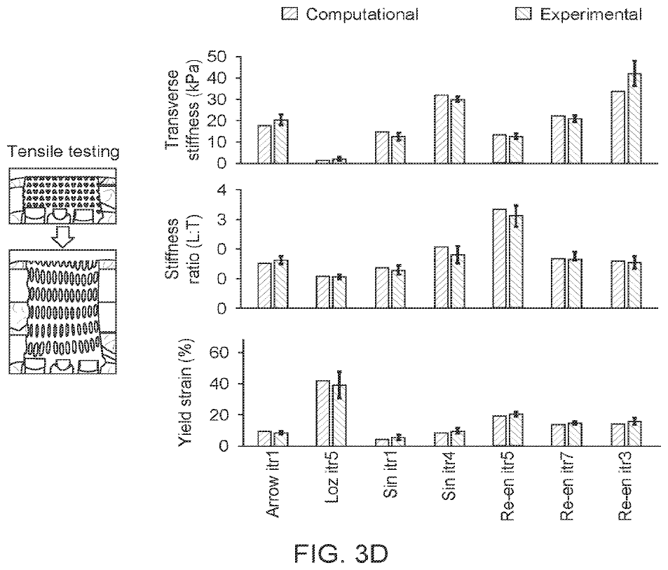

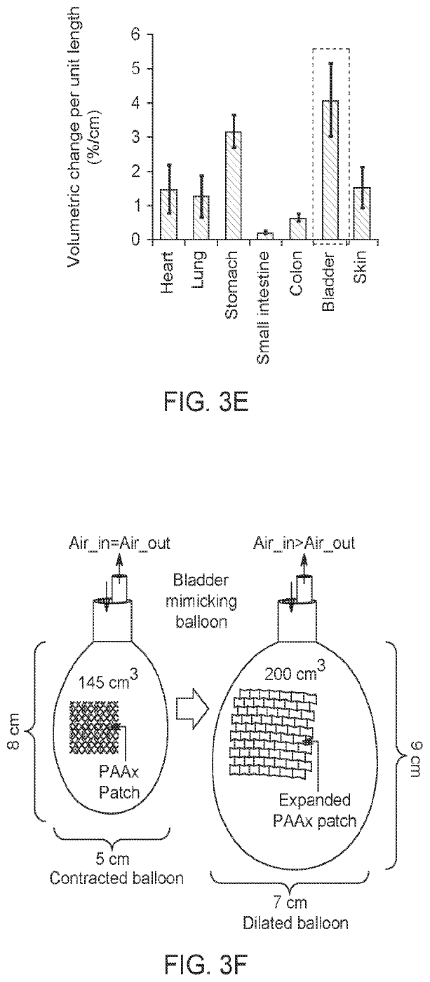

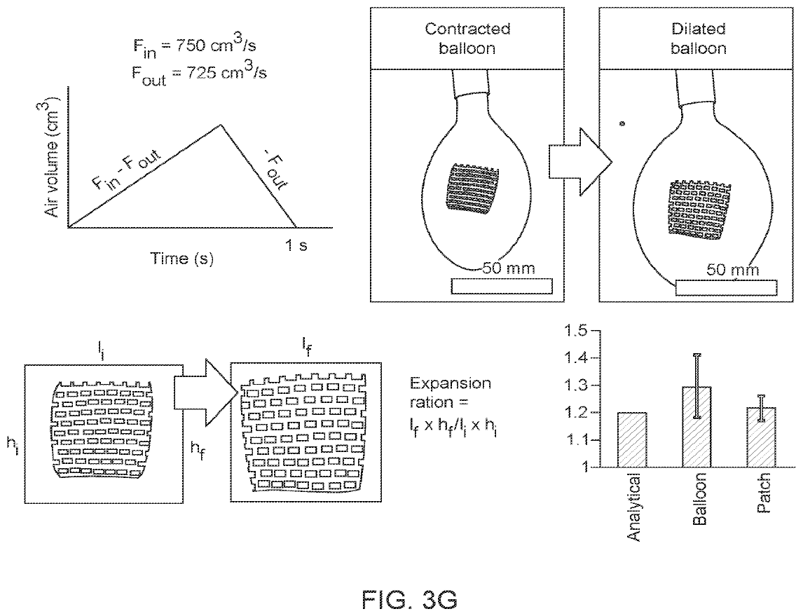

Different organs and organ pathologies require patches with different auxetic properties. To identify the optimal patch architectures and their iterations to mimic different organ dynamics, the stiffnesses and Poisson's ratios of different organs were mapped in Ashby plots, as illustrated in FIG. 3A, where patch architectures matching the appropriate organ dynamics are marked within the plot. In the plots maximum ratios were used, i.e., the ratio >1 in the y: x or x: y directions to easily compare different patch architectures, irrespective of their orientation, and easily orientate the patches to target organs. For example, in the Sin itr4 architecture, the higher stiffness direction will be oriented towards the transverse direction of the myocardium to mimic the transverse: longitudinal stiffness ratios of the myocardium. To further select specific patches matching the stiffnesses of different organs a second plot depicting bands of interest of different tissue stiffnesses along the y-axis and patch architectures and their iterations along the x-axis was created as shown in FIG. 3B.

Such an approach enables the finalization of a one patch architecture and its iteration for each investigated organ. These architectures, their iterations, and the corresponding PAAx patches suitable for different dynamic organs are shown in FIG. 3C. After selecting the most appropriate patch design for the different organs using the computational models, tensile testing of the selected patch geometries was performed to validate the accuracy of the computational modeling predictions. There was a close correlation between the computational estimates and the experimental outcomes, thereby validating the model accuracy and the robustness of the design criteria for creating patches to mimic different organ mechanics as illustrated by FIG. 3D.

Next whether patches conformed to the changes in organ shape was tested by quantifying the change in organ surface area, which directly correlates with the change in patch surface area. Since each organ has a distinct shape that varies throughout the entire organ, volumetric change per unit length of the organ was used as the rational criterion for determining the change in organ surface area as illustrated by FIG. 3E. Since the bladder undergoes the highest mean surface area change (˜4% volumetric change/cm), it was determined that the patches must conform to the mechanics of the bladder to be deemed flexible enough to withstand the most demanding organ mechanics. A mechanized setup was developed to inflate and deflate a bladder-mimicking balloon as shown in FIG. 3E. Attaching bladder-mimicking Re-en itr5 patches onto the balloon model and studying the patch mechanics showed that the patch easily conformed to the expansion and contraction of the balloon with an expansion ratio comparable to that of the balloon as illustrated in FIG. 3G. As will be appreciated, FIG. 3G shows the setup for the balloon model, which allows a constant output of air (Fₒᵤₜ) while the input (Fᵢₙ) is actuated in cycles, causing the balloon to dilate and contract, thereby stretching the patch. A comparison of the surface area of the patches demonstrates the same expansion ratio as the balloon

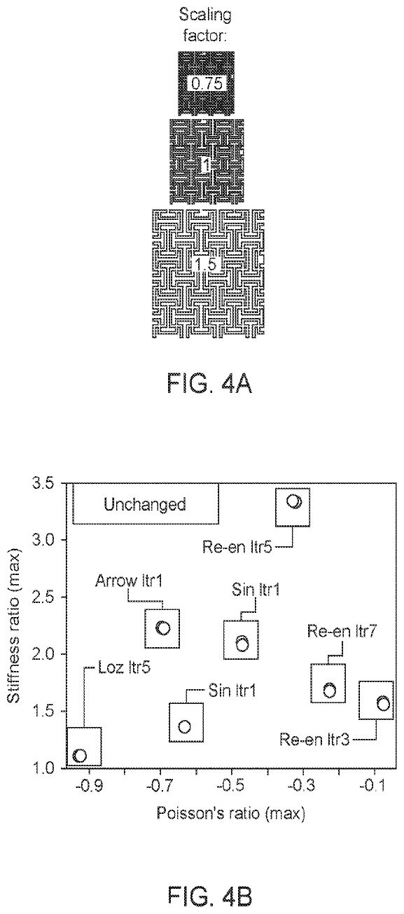

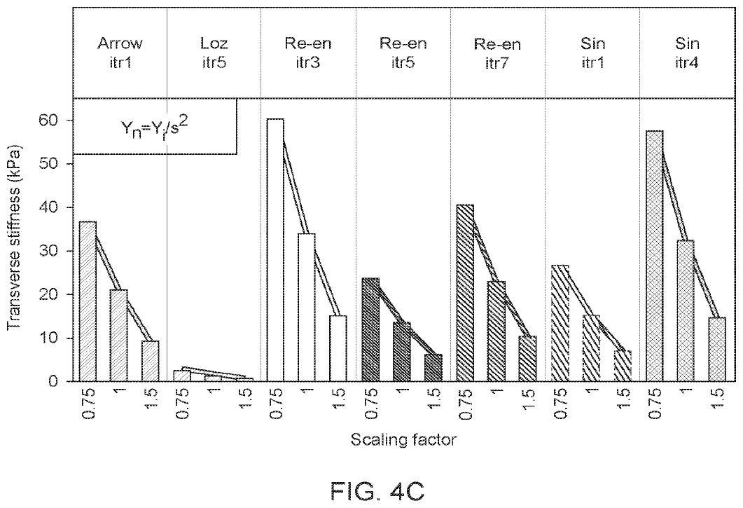

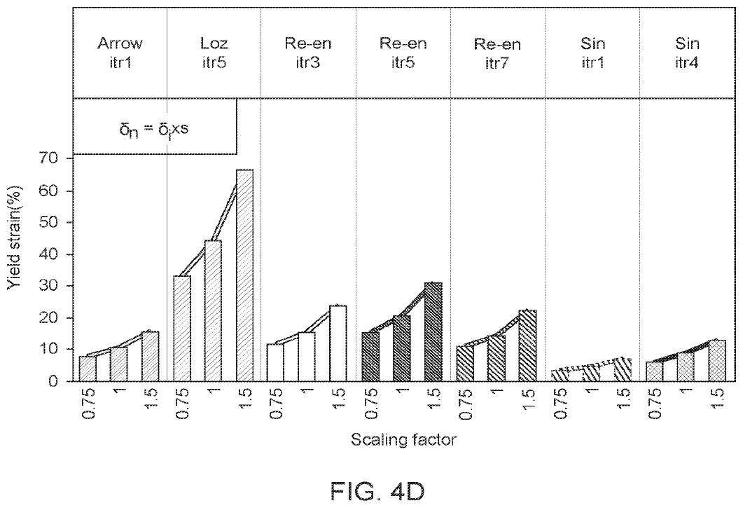

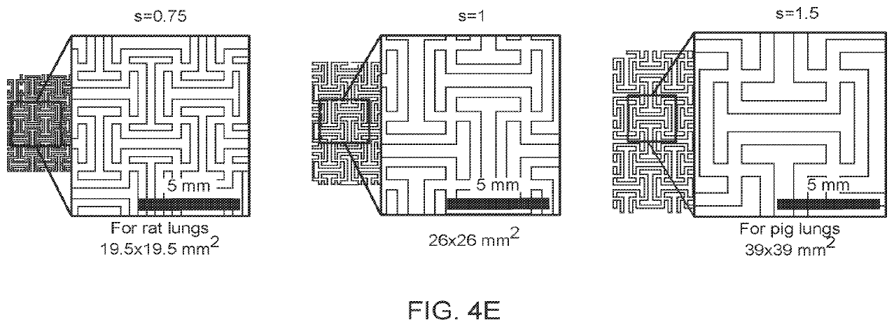

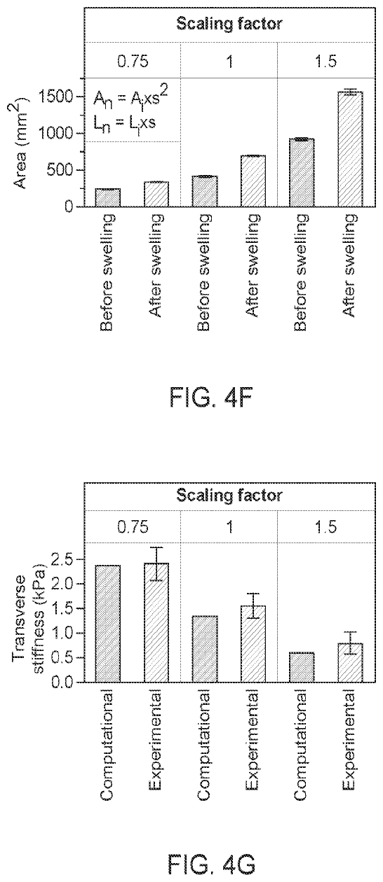

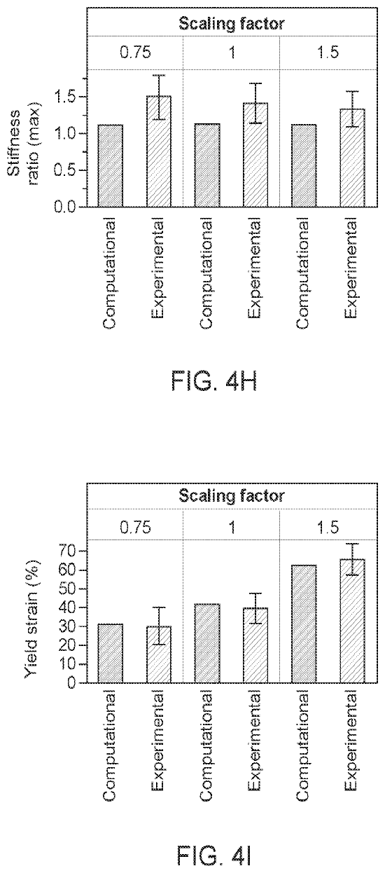

PAAx patches are scalable and can have predictable mechanical properties. In practice, biomimetic patches must account for the size of the pathology and the animal species for which the patches are developed. Organ properties such as stiffness and deformation may differ substantially depending on the species. For example, cardiac tissue stiffness is 50-300 kPa in humans but only 10-50 kPa in rodents. Accordingly, the design must be considered a priori to ensure that the patch mechanics match those of the target organs. In accordance with the technology described herein in presented an investigation of patch mechanics based on the scaling factor, which can be used as a framework to determine what changes are expected in the patches depending upon the size of the pathology or the animal model. The effects of uniformly scaling the dimensions of patches was investigated by a scaling factor s=0.75, 1, and 1.5, on PAAx patch stiffness and Poisson's ratios, and transverse stiffness (Y) and yield strain (8) of the patches as illustrated in FIG. 4, showing scalability of auxetic patches. For example, FIG. 1 shows the mechanical properties of the patches were explored for scaling factors s=0.75, 1 and 1.5 corresponding to 15, 20, and 30 mm length, respectively. As expected, the stiffnesses and Poisson's ratios of the patches remained unchanged regardless of the scaling factor as shown in FIG. 4B, since the relative ratios of the different architecture dimensions remain the same if patches are uniformly scaled across all dimensions. As a result, irrespective of size, the patch architectures mimic the anisotropic stiffnesses and Poisson's ratios of the organ. As per the computational models, the new stiffness is inversely proportional to the square of the scaling factor illustrated by Equation 1 here and further FIG. 4C.

$${Y}_{n} = {Y}_{i} / {s}^{2} \qquad ( 1 )$$

As a result, after scaling up, the patch stiffness can reduce substantially, becoming softer but stretchy. This also reflected in the yield strain (8) of the patches, which is proportional to the scaling factor, as shown by Equation 2, and illustrated by FIG. 4D.

$${δ}_{n} = {δ}_{i} \times s \qquad ( 2 )$$

Overall, these results show that, while the Stiffness and Poisson's ratios remain unchanged during uniform scaling, the scale-up patches are softer and have higher stretchability, and the scaled-down patches are stiffer and have lower stretchability. These considerations have a substantial impact on patch design depending on the target organ and species, and could be used in pre-clinical evaluation as a framework to account for the changing patch mechanics depending on the animal species in consideration.

The effect of the scaling factor after swelling of the patches was also investigated. Interestingly, expansion did not affect the relationship between the scaling factor and the size (L) of the swollen patches, i.e., the relative dimensions of the patches that expanded due to swelling were proportional to the scaling factor as shown by Equation 3. For example, 20 mm patches measured 26 mm after swelling, so for a scaling factor of 1.5, the patches should become 39 mm in size (26×1.5, illustrated in FIG. 4E). For a scaling factor of 0.75, the patches should become 19.5 mm in size (26×0.75). This was also verified experimentally in terms of patch area (Loz itr5 design, illustrated in FIG. 4F), which was proportional to the square of the scaling factor shown by Equation 4.

$${L}_{n} = {L}_{i} \times s \qquad ( 3 )$$

$${A}_{n} = {A}_{i} \times {s}^{2} \qquad ( 4 )$$

For the lung-mimicking Loz itr5 design, the computational model results were validated using tensile testing. The results for the transverse stiffness shown by FIG. 4G, stiffness ratio shown by FIG. 4H, and yield strain shown by FIG. 4I, for different scaling factors closely correlated with the computational predictions. Therefore, smaller patches (s=0.75, L=19.5 mm) were used for rat organ experiments (ex vivo and live anesthetized rats) and larger patches (s=1.5, L=39 mm) were used for pig organ experiments (ex vivo, FIG. 4).

PAAx patches conform to organ dynamics better than non-auxetic patches. Current hydrogel- or fabric-based patches do not expand and adapt to volumetric changes of dynamic organs, thereby exerting undue stresses and adverse effects on the native organ and compromising therapeutic outcomes. The patches, or PAAx patches provided herein are designed to match the native organ stiffness ratios, Poisson's ratios, and stiffnesses of the layer in contact with the patches to achieve high adaptability and compatibility to the organ dynamics. These patches can be potentially used for a wide variety of applications, such as the delivery of cells, peptides, exosomes, inorganic materials, etc. Some of these applications have been explored including the encapsulation of differently labelled MSCs in each layer of the GelMA, sustained release of mCherry peptide from the dialdehyde-modified patches, or the release of exosomes from MSCs encapsulated within the patches with good results.

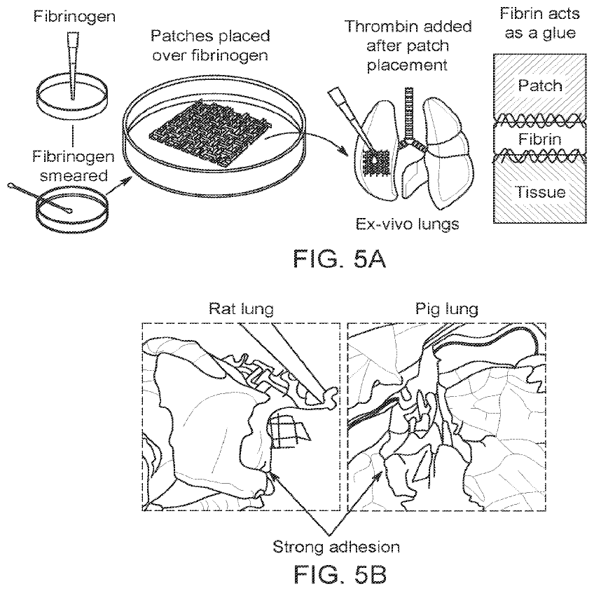

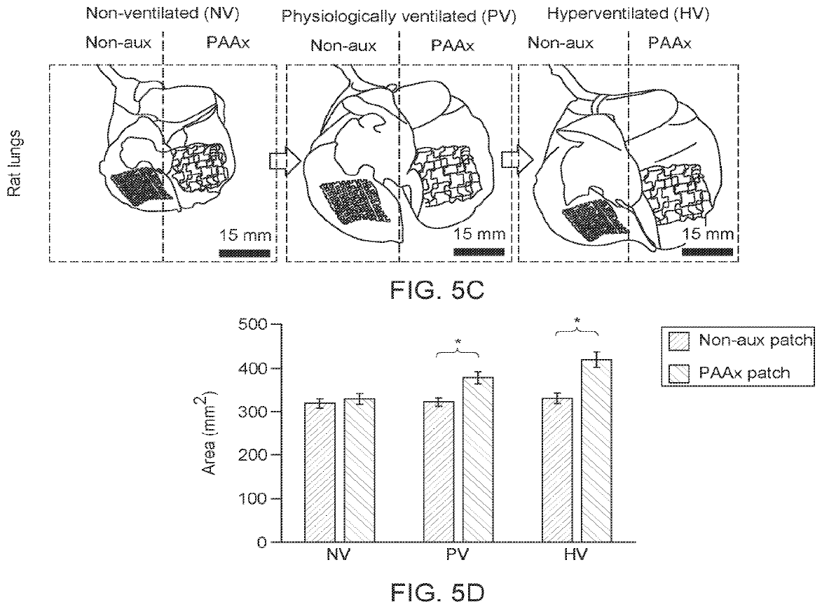

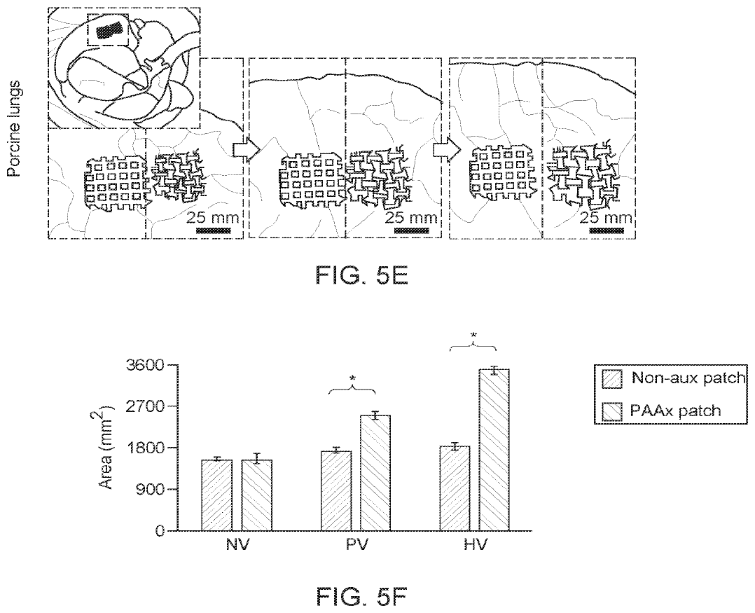

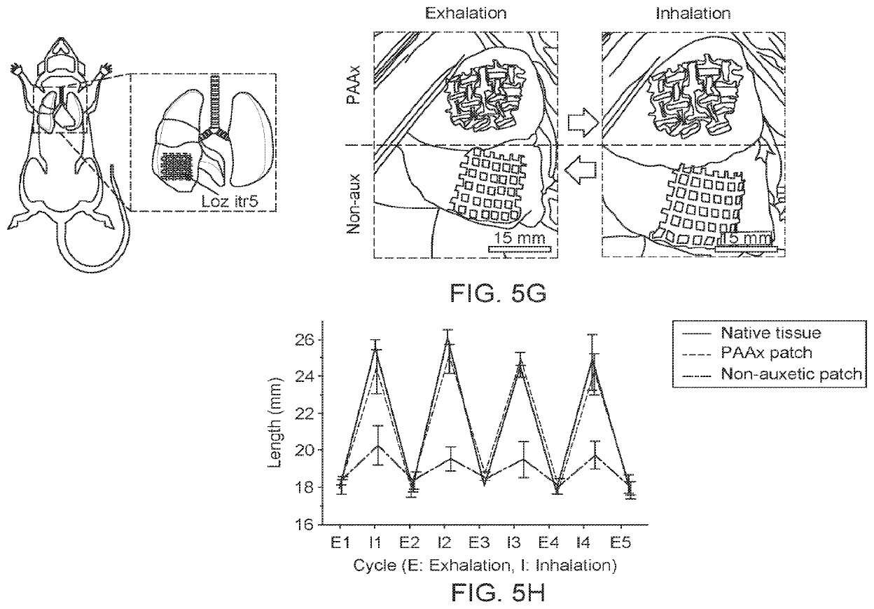

The ability of the PAAx patches to conform to the organ mechanics (contraction and expansion) ex vivo on rat and pig lungs was also tested. FIG. 5 illustrates a comparison of auxetic and non-auxetic patches in ex vivo rat and pig lungs and in live anesthetized rat lungs. Patches were adhered to the organs using a thin layer of fibrin glue as illustrated in FIG. 5A to facilitate strong adhesion of the patches to the organs as illustrated by FIG. 5B. PAAx patches were compared with conventional, non-auxetic patches such as those featuring hexagonal or square holes. The scaled-up patches (s=1.5, FIG. 4E) were used for pig lungs, while the scaled-down patches (s=0.75) were used for rat lungs. Unlike conventional patches, the PAAx patches expanded during lung expansion under physiological ventilation and hyperventilation, allowing adaptation to the volumetric changes of the lung as illustrated in FIG. 5C and FIG. 5E. As a result, as the lung expanded during physiological ventilation (PV) and hyperventilation (HV), the PAAx patches displayed increased surface area by approximately 25% (PV) and 40% (HV) for rat lungs, and approximately 62.5% (PV) and 210% (HV) for pig lungs (p<0.05, FIG. 5D and FIG. 5F), posthoc analysis via Tukey's HSD), respectively. In contrast, conventional non-auxetic patches contracted in one dimension when stretched across another, so the surface area did not increase (p>0.05, posthoc analysis via Tukey's HSD).

The patches (scaled-down, s=0.75, Loz itr5 patch) were then applied using thin layer of fibrin over the lungs of live anesthetized rats undergoing terminal open chest surgery (FIG. 5G). After application, the PAAx patches demonstrated compliance comparable to the native lung across all physiological expansion-contraction cycles, while the non-auxetic patches could only demonstrate limited compliance as illustrated in FIG. 5H, showing that after administration over the organ, the auxetic patches demonstrated similar deformation to the native tissue during different inhalation-exhalation cycles of the lung.

PAAx patches can be used to prevent pulmonary air leaks. Injuries of dynamic organs that involve fluid (e.g. hemorrhages) or air leakage (e.g. pneumothorax) are particularly challenging. The volumetric deformation of the organs warrants the use of auxetic patches to seal the leakage, so that the patches can allow physiological deformation of the organs. In this study in accordance with the technology an emphasis was placed on re-designing the PAAx patches to be able to reduce pulmonary air leaks induced through a puncture wound in a lung of an anesthetized rat under open chest surgery. The objective was to seal a defect in the visceral pleura and lung parenchyma in the open chest. Such an injury or spontaneous occurrence in a closed chest can lead to a pneumothorax. Conventional methods for lung sealing involve the use of blood pleurodesis, colloquially termed blood patches, but this procedure can be complicated by infection and empyema. Other studies have used hydrogel-based plugs for the treatment of lung punctures; for example, an adhesive hydrogel composed of dopamine-functionalized alginate methacrylate and gelatin as a pleural sealant. In these rat models with puncture wound induced through needle (18G) injury, the restoration of tidal ventilation pressure was not quantified and there was no comparison with other sealants such as fibrin glue. Another study developed a polyglycolic acid sheet and alginate gel to reduce pulmonary air leaks. Although the patches enhanced sealing of the injury, their administration required a motionless lung for 5 min. Another study developed a human protein-based sealant, which was used to treat lung puncture induced through surgical incision after thoracotomy in pigs. Although the material ensured freedom from pneumothorax, a restoration of ventilation pressure was not quantified. Of note, all these materials were homogeneous, non-auxetic, and were not specifically designed to match the stiffness and mechanics of the lung tissue. As a result, even after successful sealing of injury, the lung function may be inhibited as the patches can physically restrain the lungs from expanding volumetrically. To address these shortcomings, the present adaptive lung-mimicking PAAx patches in some instances can be tailored for the treatment of pulmonary air leakage.

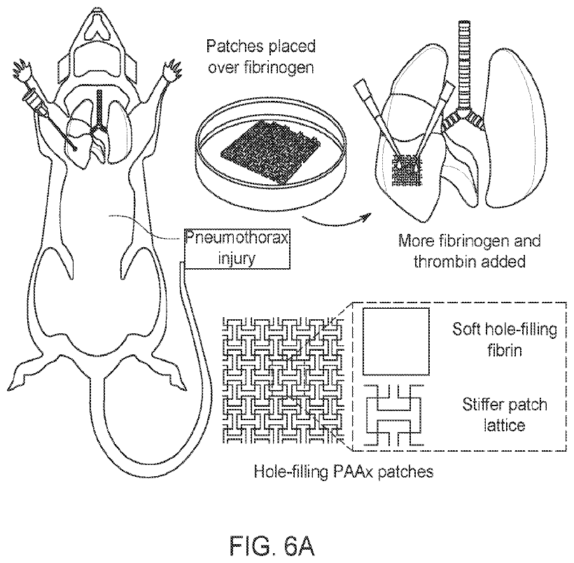

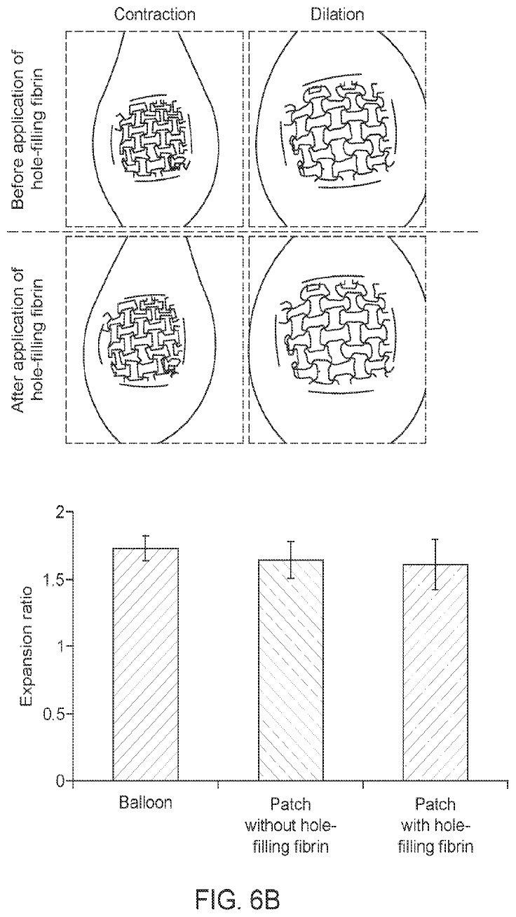

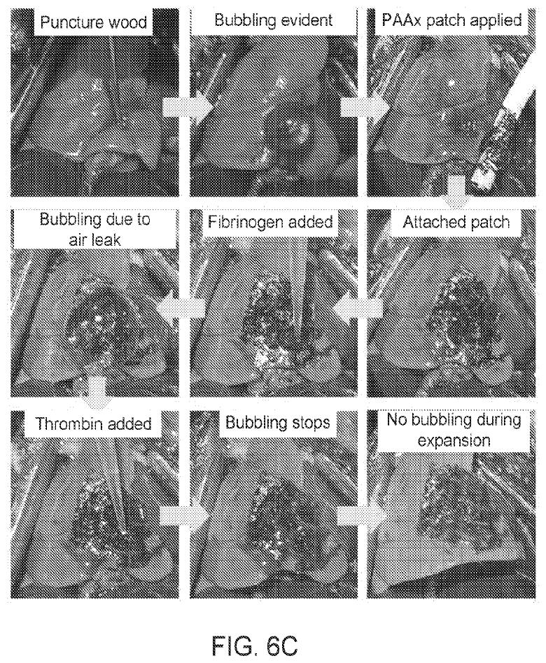

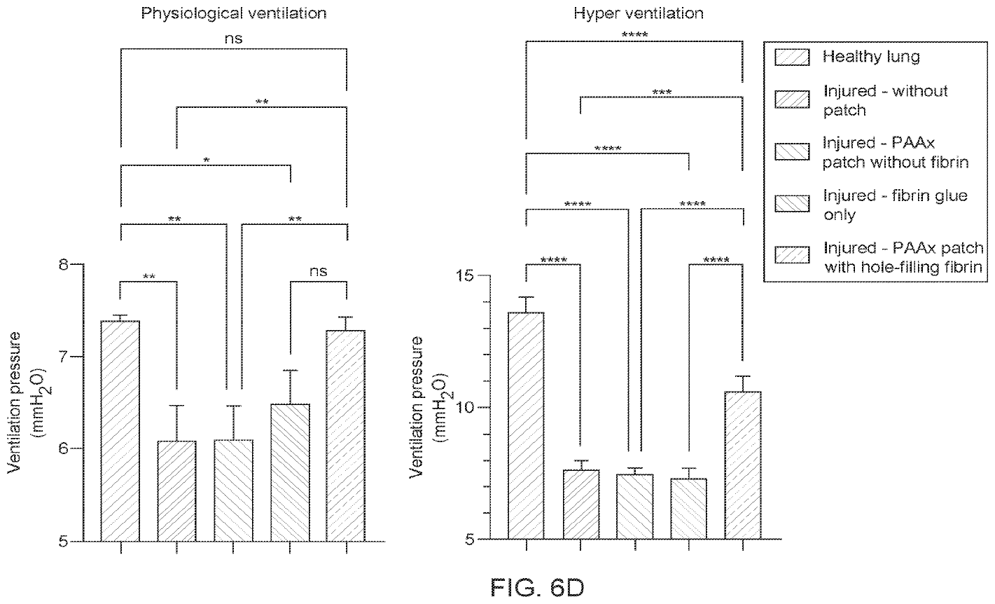

A challenging aspect with sealing wounds in dynamic organs is that that there should virtually no voids present in patches, to prevent any fluid leakage, which is difficult to achieve since the auxetic properties are derived through the expansion-contraction of the voids in the auxetic architectures. The present generation of PAAx patches can be modified by combining them with soft fibrin glue to create hole-filling PAAx patches (illustrated in FIG. 6A), where the fibrin glue fills the space between the lattice elements of the patches and allows for adhesion of the patches to the organ. Since the fibrin glue is soft and stretchy with an elastic modulus of <1 kPa, it allows the patches to fully exhibit their auxetic property while preventing air leakage from the patch area. This is evident in the balloon experiments of patches with and without hole-filling fibrin as illustrated in FIG. 6B, which demonstrates that the patch expansion is not affected due to the presence of the fibrin. In the rat model shown in FIG. 6C, the patch was applied over surgically induced injury, the fibrin prepolymer (fibrinogen+proteolysis inhibitor, TISEEL, Baxter) was applied over the patches, and thrombin added. This way, the fibrin filled up the voids between the lattice elements and prevented air leakage from the lung, while allowing the patch to expand with the lung. Here it is confirmed that an auxetic patch has been and can be used for the treatment of a pathology involving air or fluid leakage.

During testing of the sealing effect of the hole-filling PAAx patches, lung puncture was induced using a 18G needle. The ventilation pressure was measured in healthy and injured lungs with or without various treatments—fibrin-only, PAAx patches without fibrin (patch-only) and PAAx patches with fibrin—using a pressure transducer (Transpac IV, ICU medical). In these experiments, separate rats (n=3 per treatment) were tested under both physiological ventilation (tidal ventilation volume of 7.5 ml/kg) and hyperventilation states (tidal ventilation volume of 12.5 ml/kg). FIG. 6D shows the results of the dilation ventilation pressure (measured using a pressure gauge attached at the airway outlet) before and after injury and treatment. FIG. 6D illustrates variation in tidal pressure of healthy or injured lungs under physiological ventilation (PV, 7.5 ml/kg ventilation volume) and hyperventilation (HV, 12.5 ml/kg ventilation volume). Administration of the PAAx patches with the hole-filling fibrin completely restored the lung function during PV, and up to 80% lung function during HV, while other treatments did not result in any improvement in tidal pressure of the lung. *p<0.05, **p<0.01, ***p<0.001, ****p<0.0001 by one-way ANOVA followed by posthoc analysis via Tukey's HSD.

Under physiological ventilation, the ventilation pressure dropped from ˜7.4 mm H₂O to ˜6.4 mm H₂O. Herein, the treatment using either fibrin or patches alone was unable to restore the pressure, because the fibrin did not persist at the injury zone in the fibrin-only groups, and because the air was still able to leak in the patch-only groups. In contrast, the PAAx patches administered in conjunction with the hole-filling fibrin clearly demonstrate the capability to restore the physiological ventilation pressure back to that before injury. Under hyperventilation, the tidal ventilation pressure was ˜13.8 mmH₂O and ˜7.6 mmH₂O before and after lung injury, respectively Similar to the physiologically ventilated rats, the fibrin-only and patch-only formulations did not demonstrate any improvement in airway pressure. In contrast, the hole-filling patches resulted in a significant improvement in airway pressure and restored airway pressure by up to 80% (˜11.1 mmH₂O) of the original tidal ventilation pressure. Further, the differences between fibrin-only and PAAx patch with fibrin groups are especially pronounced in the hyperventilated state, demonstrating that the patches are robust and able to withstand strenuous volumetric changes during hyperventilation. Restoration of the airway pressure during the hyperventilation state was limited by the constant motion of the lung during the application of the patches, which might have affected patch adhesion over the injury site. Nevertheless, this technology was effective at restoring airway pressure during physiological ventilation and could potentially be used an effective technique for the treatment of many injuries involving fluid or gas leakage.

The development of adaptive biomimetic patches for the repair and regeneration of dynamic organs such as the lungs, heart, bladder, stomach, intestines, and skin remains scientifically and clinically challenging. There are currently only a limited number of clinically approved patches for dynamic organs; examples include the Cor™ Patch and CardioCel® patch for cardio-vascular defects. While these patches are reparative, they are not curative, and there remains considerable scope to improve the effectiveness of patch-based therapeutics. Imparting anisotropy and auxetic characteristics to the current range of patches could substantially improve their translation potential and long-term clinical efficacy.

The adaptive biomimetic PAAx patches in accordance with the present technology can be used for a wide variety of therapeutic applications and can also be loaded with biologics. To support this information, different material formulations were prepared and investigated that could allow encapsulation and fine-tuning of the release of therapeutics (such as cells, peptides, and exosomes). In case a different material with different stiffness and yield strains is used for the patch, the patches can still be made adaptive to the target organ through the design guidelines shown below. For example, for a lung patch (stiffness <4 kPa) with a thermoplastic-based material (stiffness >200 kPa), the dimensions of the lattice elements (h, d, t, etc.) in lozenge or chiral truss architectures can be changed such that the stiffness is substantially reduced compared to the bulk material while keeping the Poisson's ratio close to −1 (close to that of lung tissue).

In rat studies, the scaling-down of the patches resulted in stiffness ˜2.5 kPa, which is similar to that of rodent lungs the scaling-down of the patches resulted in stiffness ˜2.5 kPa, which is similar to that of rodent lungs. While in the present case, the lung function was not impeded upon scaling down of the patches, a patch system designed to mimic human organs may provide sub-optimal results if, after scaling, the patch stiffness is significantly higher than the native tissue stiffness. Such a patch may need to be softened by changing the material composition (reducing material concentration or degree of substitution) or processing conditions (UV exposure duration).

Finally, patch implantation is a very important consideration that could govern translational potential. Compatibility with minimally-invasive video assisted thoracic surgery (VATS) or robotics will improve patient acceptance and post-surgical recovery. To further improve patch application, it is possible to impart intrinsic bioadhesiveness through chemical modification of the patch materials, or by imparting bioadhesive architectures within the auxetic patches. Fibrin or other materials could also be applied to the surface of a partially inflated lung before applying the patch.

Based on the present technology, design guidelines can be developed, noted here, to allow any new patch system to conform to the organ dynamics: (1) The anisotropic mechanics of the patches can be made to match those found in the native tissues by matching their stiffness ratio and Poisson's ratios, respectively, as these properties are not material specific. (2) For materials which are stiffer than the target organ tissue, appropriate tuning or scale of the patch design, or re-thinking of the material formulations, should be carried out to match the patch stiffness with that of the organ and to prevent undue stresses on the organ tissues. (3) The patches should exhibit a linearity of the stress-strain curve up to the max strain of the organ. This will allow the patch to behave as an elastic material, allowing a complete recovery of the mechanical characteristics (stiffness, Poisson's ratio) during the expansion and contraction cycles of the organs. As an example, since cardiac tissue undergoes a max linear strain of up to 10%, cardiac patches should exhibit linearity in the stress-strain curve up to at least 10% strain. (4) At the max strain, the internal stresses within the patches should not exceed the yield strength of the material to ensure that the patch does not break during physiological expansion of the organ. (5) The patch mechanics should be tailored to the animal model in use during pre-clinical studies. (6) For organs such as the lung which feature Poisson's ratios close to −1 (i.e., the organ undergoes the same expansion both longitudinally and transversally), patches based on lozenge truss and chiral truss architectures may be more appropriate. The design of the lattice elements in these architectures is such that it allows the patch to undergo almost the same deformation transversally when the patch is stretched laterally. (7) For organs such as the bladder or the heart, which feature high stiffness ratios, the re-entrant honeycomb, arrowhead, or sinusoidal ligament architectures are more appropriate, since these architectures can be made to feature different design of the lattice elements across different directions, thereby imparting anisotropy.

Accordingly, the present technology is directed to and enables the development of a new class of photocrosslinkable patches with a cell adhesive GelMA bottom layer and a non-adhesive PEGDA top layer. Further this highlights how these patches can be appropriately tailored to conform to the mechanics of different organs by introducing anisotropic and auxetic characteristics within the patches, and how scaling affects the mechanical properties of the patches. The novel hole-filling PAAx patches demonstrated enhanced reduction of pulmonary air leakage compared to fibrin glue only. These results pave the way for translation of hole-filling PAAx patches for dynamic organ pathologies. The rational design principles demonstrated here can inspire a new generation of patches that cater to the demands of dynamic organs and improve their clinical translation.

Materials synthesis. Methods for methacrylation of gelatin were used to derive GelMA with 40% methacrylation (verified by NMR analysis). PEGDA (6 kDa, 100% acrylation) was bought from Volumetric Inc. (P6000-P-1; Houston, TX). The lyophilized materials were dissolved in phosphate buffered saline (PBS) at 10% w/v (GelMA) or 20% w/v (PEGDA). Further, the photoinks were constituted by adding 0.03% w/v lithium phenyl-2,4,6-trimethylbenzoylphosphinate (LAP) photoinitiator and 0.015% v/v FD&C yellow (food dye) as the UV absorptive component.

Patch printing and postprocessing. To fabricate the bottom layer of the patches, 500 μl of GelMA was liquefied at 37° C. was added to a rectangular enclosure (30×30 mm²) fixed to the top of a PDMS-coated petri dish. The liquefied layer was uniformly smeared across the vat using a plastic blade and allowed to thermo-reversibly crosslink for 5 min at 24° C. (ambient temperature). Next, the top PEGDA layer was added at 24° C. The bilayer setup was then placed above a projection system (LumenX, Cellink AB) and selectively photocrosslinked through bottom-up projection via a digital micromirror device (DMD) for 2 minutes at 405 nm and 40 mW/cm². In the presence of photoinitiator (LAP), the covalent crosslinking of methacrylate groups in GelMA in the bottom layer, the acrylate groups in PEGDA in the top layer, and between the methacrylate and acrylate groups across the two layers led to the formation of a robust photocrosslinked network. Further, the presence of UV absorptive dye in each layer prevented diffusion of UV light to allow high-resolution fabrication. The vat with the construct was then transferred to an incubator at 37° C. to liquefy the non-photocrosslinked GelMA. The patches were then washed extensively with warm (37° C.) PBS to remove any non-photocrosslinked material and then dipped in 10 ml of PBS for up to 3 h at 37° C. to allow complete swelling of the patches prior to any experimentation.

Tensile testing. Tensile tests were performed in a novel developed setup. The setup comprised: 1. a linear stage actuator (101-80-124, SainSmart) driven using a stepper driver (101-60-197, SainSmart) and a programmable motion controller (101-60-199, SainSmart) to stretch the patches, and 2. a 5 kg load cell (TAL220B, Sparkfun) and amplifier (HX711, Sparkfun) combo used as the stationary anchor for the patches. The ends of the patches were fixed to the load cell or the movable platform of the linear actuator, respectively, using 50 μl cyanoacylate glue (Vetbond, 3M). Patches were strained at a constant rate of 0.125 mm/s, consistent with existing studies, and the tensile stress vs the strain was plotted in the serial plotter window of the Arduino interface to determine the mechanical properties of the patches.

In vitro evaluation of cell attachment, viability, and metabolic activity in the presence of patches. NIH 3T3 fibroblasts (CRL-1658, ATCC) were cultured in Dulbecco's modified Eagle medium (DMEM) and 10% fetal bovine serum and 1% penicillin-streptomycin until 80% confluency, after which the cells were passaged using 0.25% trypsin-EDTA and centrifuged at 500 g for 5 min to derive the cell pellet. The pellet was re-suspended in fresh medium at a concentration of 2×10⁵ cells/ml. To evaluate cell attachment over the patches, the patches were placed within non-culture treated six-well plates with either the GelMA or PEGDA layer facing upwards (n=3 per group). 2 ml of the cell suspension was then added on top, and the plates incubated overnight and evaluated the following day for cell attachment using Calcein-AM dye (65-0853-39, Thermo Fisher Scientific, Waltham, MA). For viability assessment, the cells (2 ml suspension/well) were added to the six-well plates first and allowed to attach for 6 h followed by adding the patches to the wells. The cells were then cultured up to 5 days, with media changes every day. Every day, the wells were evaluated for viability using the Live/Dead™ cytotoxicity kit (L3224, Thermo Fisher Scientific). For metabolic activity assessment, 10,000 cells were added into a transwell insert (Day 0) in a 24-well plate and 100 μl of medium added on top. The patches were cut to 5×5 mm² and placed underneath the transwells with 600 μl of medium. On days 1, 3, and 5, the inserts containing cells were transferred to empty wells and the supernatant medium replaced with 120 μl of MTT assay reagent (prepared as per the manufacturer's protocol, 11465007001, Millipore Sigma, St Louis, MO) before incubation for 4 h at 37° C. 100 μl of the MTT assay was then collected from the wells and replaced with fresh medium after washing the cells twice with PBS. The medium in the bottom wells containing patches was also replenished with 600 μl fresh medium and the transwell inserts transferred back to the bottom wells. The absorbance of the collected MTT reagent (100 μl) was measured at 570 nm.

Patch Design. The patches were designed in Solidworks (Dassault Systems, Vélizy-Villacoublay, France). First, the repetitive elements (see e.g. inset of FIG. 2A) of the patch lattice were designed, followed by defining a linear array projection to generate multiple repetitive elements across both the horizontal and vertical directions. An extruded cut of this array was made through a rectangular block (20×20×1 mm³) to generate the patch architectures. The different dimensional attributes (w, h, r, etc.) in the repetitive elements were assigned as global variables, which allowed altering the variables to change the overall patch design.

Computational modeling of the patch mechanics. The computational model was developed in the structural mechanics module of COMSOL Multiphysics (COMSOL Inc., Stockholm, Sweden). The boundary conditions included a fixed domain on one end of the patch and a linear displacement of 10% strain on the other end. The patches were set to stretch along either the x or y axis at a time. To derive accurate results, a meshing density of 1/10^{th} the size of the minimum dimension was selected, in accordance with previous studies. Along each direction (longitudinal and transverse) of stretching, the stiffness of the patches (Y) along the longitudinal or transverse direction was determined as a function of strain energy (U, output of COMSOL), strain (s=10%) and volume of the patch (V) as per the following relationship:

$$Y = {s}^{2} V / 2 U \qquad ( 5 )$$

Consequently, the stiffness ratio of the patches was determined as the ratio of the longitudinal to the transverse stiffness. The Poisson's ratio (v) is determined as a function of strain(s) and deformation along the orthogonal direction (4) as follows:

$$v = Δ / s \qquad ( 6 )$$

Due to intrinsic anisotropy within some of the patch architectures, the stiffness can be different along the longitudinal or transverse directions. Herein, the max stiffness ratio was >1, where the higher stiffness component was kept in the numerator. In addition, the Poisson's ratio can also be different in the longitudinal and transverse directions. In this case, the greater magnitude of the ratio was considered as the maximum Poisson's ratio. The yield strain of the material is defined as the strain at which the max von Mises stress in the internal structure of the patches exceeds the yield strength of the material. Since the maximum internal stress varies linearly with the amount of strain (determined from the computational model), the yield strain (δ) was determined as a function of the max internal stress (σₘₐₓ) at any given strain(s) and the yield strength of the material (Yₛ˜25 kPa, determined in FIG. 2C) as follows:

$$δ = {Y}_{s} s / {σ}_{max} \qquad ( 7 )$$

Balloon model mimicking bladder mechanics. The balloon model setup was developed in-house. It consisted of a programmable linear actuator capable of rotating a valve that supplies compressed air to a balloon. The rotation of the air valve was calibrated using an air flow meter (VFA-26-BV, Dwyer Instruments) at the valve outlet, such that a flow rate of 750 cm³/s was achieved at the balloon inlet. To allow air release from the balloon, a hollow pipe was attached at the neck of the balloon and the pipe diameter calibrated using another air flow meter such that an outlet air flow of 725 cm³/s was achieved. During experiments, the patch was placed over the balloon and attached using 5 μl of cyanoacrylate glue administered at each vertex of the patch. The calibrated system was used to achieve repeatable expansion-contraction of the balloon to mimic the bladder dynamics (see Video S1). Note that a bladder would be twice the size of the balloon used in these experiments and, as such, will reach up to 400 ml in capacity when expanded.

Ex vivo evaluation of patch compliance. Porcine lungs were obtained from North Carolina State University School of Veterinary Medicine and were cold flushed after euthanasia. The rat lungs were excised after euthanasia under anesthesia (UNC Chapel Hill Institutional Animal Care and Use Committee (IACUC) Protocol No: 20-045.0). The patches were attached onto excised lungs using fibrin sealant (TISEEL, Baxter). The sealant was prepared as per the manufacturer's guidelines, wherein the fibrin pre-polymer solution was prepared by mixing fibrinogen powder with fibrinolysis inhibitor solution (available in the manufacturer's package) at 170 mg/ml, and the thrombin crosslinker solution was prepared by mixing thrombin powder and calcium chloride solution (available in the manufacturer's package) at 50 mg/ml. To coat a thin layer of fibrinogen on the GelMA side of the patches, 50 μl of fibrin prepolymer was added to a 35 mm diameter petri dish, followed by smearing it throughout the petri dish using a cotton swab and placing the patches on the petri dish with the GelMA side facing down. Then, after 30 seconds, the patches were transferred to the organ (GelMA side still facing down) and 50 μl of thrombin solution added on the top immediately. Physiological ventilation was provided at a tidal volume of 7.5 ml/kg and a rate of 70 breaths per minute. Hyperventilation was induced by increasing the tidal volume to 12.5 ml/kg while lowering the rate to 40 breaths per minute to maintain consistent minute ventilation.

Evaluation of patch compliance in live anesthetized rat models. Rats (UNC Chapel Hill IACUC Protocol No: 20-045.0) were anesthetized with an intraperitoneal injection of ketamine/xylazine and underwent tracheotomy and ventilation with isoflurane and oxygen. A sternotomy was performed to expose the lungs. A tidal ventilation volume of 7.5 ml/kg was used for these live animal experiments. The protocol for patch administration was the same as that for the ex vivo experiments. In these experiments, the auxetic (Loz itr5) and non-auxetic patches were placed on the lungs.

Measurement of patch size in the balloon, ex vivo, and live anesthetized animal experiments. In the bladder-mimicking balloon and ex vivo and live animal experiments the patch motion under different cycles was captured as a video. The length and width of the patches were determined using the ImageJ distance measurement tool across different frames of the video. Patch area was determined as the multiplication of the length and width.

Lung sealing via hole-filling PAAx patches. The procedure to prepare the rats (UNC Chapel Hill IACUC Protocol No: 20-045.0) and expose the lungs for simulated puncture injury was the same as that for the patch-compliance experiments in live rats. The lungs were kept under either 7.5 ml/kg (physiological ventilation) or 12.5 ml/kg (hyperventilation) of ventilation volume. Additionally, airway pressure was monitored via a Transpac IV transducer. Based on prior work, an 18G needle was inserted up to 1 cm deep to injure the left lung or right lower lobe while under anesthesia to induce the puncture wound. The details of the experimental procedure are highlighted in FIG. 6. The patches were then applied over the injury followed by administering 50 μl of fibrin pre-polymer solution and then 50 μl of thrombin solution. Any excess bubbles still present above the patch were burst using the pipette tip. The corresponding changes in the ventilation pressure measurements were noted at each step. For the fibrin-only groups, 50 μl of fibrin pre-polymer solution and then 50 μl of thrombin solution were added directly to the injury. For the patch-only groups, the patches were applied without any fibrin.

Statistical analysis. All experimental data are presented as means and standard deviations. For all experiments (in vitro, ex vivo, and live animals), a sample size of n=3 (biological or non-biological) was used. Statistical analysis was performed in JMP® (SAS, Cary, NC) at a significance level of α=0.05 using a two-way ANOVA for the patch swelling and metabolic activity assessment (FIG. 1) and ex vivo studies (FIG. 5), and one-way ANOVA for the tensile tests (FIG. 2), balloon experiments (FIG. 3) or lung puncture studies in anesthetized rats (FIG. 6). The differences between groups were determined using Tukey's HSD Pos-hoc tests.

### Example 2—Adhesive Anisotropic Auxetic Patches for Organ and Wound Repair

According to some aspects of the present technology adhesive anisotropic-auxetic patches for wound repair are provided, for instance, elastic or ultra-elastic and/or instantly adhesive anisotropic-auxetic patches for dynamic organ and wound repair.

Most conventional patches do not adhere to tissues nor adapt to the demanding mechanics of dynamic organs such as the lungs and heart. Here, a novel hydrogel composition for patch synthesis is provided that is ultra-adhesive, highly stretchable, and printable into complex architectures. The patch designs were based on in silico modeling of over 60 auxetic and anisotropic designs that feature a broad range of stiffness, stretchability and Poisson's ratios. The patches were based on a novel photocrosslinkable hydrogel composition that imparts: (1) compatibility with rapid projection printing (<2 min fabrication time) at high resolution (up to 375 μm minimum feature size), (2) ultra-stretchability (stretching to >300% its original length without losing elasticity), (3) the ability to adhere instantaneously (<1 s) and strongly (2.5-fold stronger than commercial fibrin glue) to wet and dry tissues, and (4) the ability to conform to different dynamic organs. Additionally, the patch biocompatibility, conformation, and robust wound healing capability in vivo is demonstrated. Further, a novel strategy to create an auxetic hole-filling patch designed for the treatment of puncture wounds in dynamic organs, such as the lung is demonstrated.

Dynamic organs such as the lungs, heart, stomach, bladder, intestines, and musculoskeletal tissues present specific challenges towards the effective use of patch-based therapeutics. Their repetitive physiological motion creates a fatigue loading environment that predisposes the patch to premature breakage or detachment. Patches are typically held in place using sutures, staples, or surgical glues that can similarly detach or strain the organ over time and require specialist dexterity to apply. Moreover, these procedures can cause toxicity, inflammatory responses, undesirable matrix remodeling and scar tissue formation, and post-operative pain. While intrinsically bioadhesive materials exist, most require at least several minutes of tight interfacing between the tissue and patch for effective bioadhesion, making them unsuitable for attachment to dynamic organs in the clinical setting. More importantly, most patches cannot adhere to internal organs covered by fluid or wet skin.

In addition to repetitive motion, there are other critical challenges associated with dynamic organ mechanics. Except for musculoskeletal tissues, other dynamic organs dilate or contract volumetrically, such that their surfaces exhibit a negative Poisson's ratio, i.e., the surface expands across multiple directions simultaneously. For example, the stomach can volumetrically dilate up to 250%, imparting a Poisson's ratio between −0.5 and −0.7, and the lungs dilate up to 40% 13, exhibiting a negative Poisson's ratio between −0.85 and −0.95. Most current patches based on biocompatible fabrics or hydrogels are intrinsically homogeneous or contain basic features such as simple porous structures, crisscrossing, or honeycomb meshes. While these architectures may be suitable for musculoskeletal patches, they cannot adapt to the auxetic characteristics of dynamic organs such as the lung or heart.

Another specific challenge posed by the dynamic organs is the intrinsic anisotropy of the distribution of cells and extracellular matrix fibers. This leads to a different organ deformation in the fiber orientation (longitudinal) direction, compared with the orthogonal (transverse) direction. Such a property is captured by the stiffness ratio, which describes the organ's relative resistance to deformation in the longitudinal and transverse directions. For example, the myocardium is 2-4 times stiffer in the longitudinal direction than the transverse direction, and the amount of deformation in the transverse direction can be almost twice that in the longitudinal direction. Most current auxetic or non-auxetic patches do not mimic these differential deformational characteristics (anisotropy) of the organs. This failure to accommodate auxetic and anisotropic properties means that most current patches are prone to premature detachment or exert undue stresses on the organ over repetitive cycles.

Together with auxetic and anisotropic properties, the large deformations of dynamic organs can also limit patch effectiveness. Any patch therapeutic must exhibit elastic behavior (linearity of the stress strain curve) throughout physiological organ cycling so that it exhibits the same mechanical characteristics throughout every cycle. For example, the stomach, bladder, and intestines can undergo up to 100% radial strain, i.e., they stretch up to twice the radius. Therefore, the yield strain of patches intended for these organs should be at least 100%, and such an ultra-elasticity can be a difficult to achieve in practice. While some multinetwork hydrogels are ultraelastic, they only have limited anisotropic and auxetic characteristics.

While attempts have been made to create auxetic and anisotropic patches for the heart and skin, these are not intrinsically bioadhesive or ultra-elastic, and patches have yet to be developed for the full range of organs or their specific pathologies. This represents a huge opportunity gap in patch design. Therefore, the objective of the present technology is to develop and provide a new biocompatible patch material for dynamic organs with the following characteristics: (i) easy and rapid printing at high resolution into anisotropic-auxetic architectures; (ii) instantaneous and strong bioadhesion; and (iii) ultra-elasticity. Patches in accordance with aspects of the present technology were rationally designed to mimic the mechanics of different organs and then tested for their conformation to the organ mechanics ex vivo and in vivo. Lastly, the patches were also evaluated with respect to their therapeutic potential in two exemplar pathologies, wound healing and pulmonary air leakage.

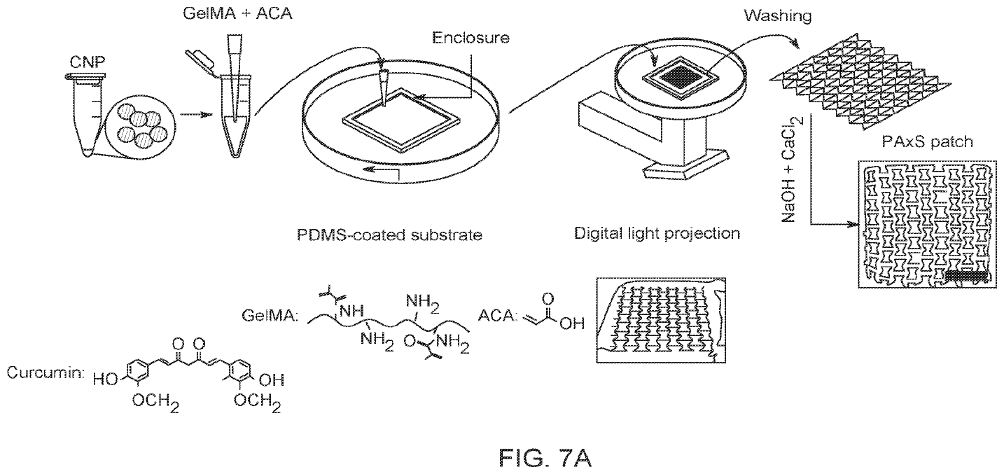

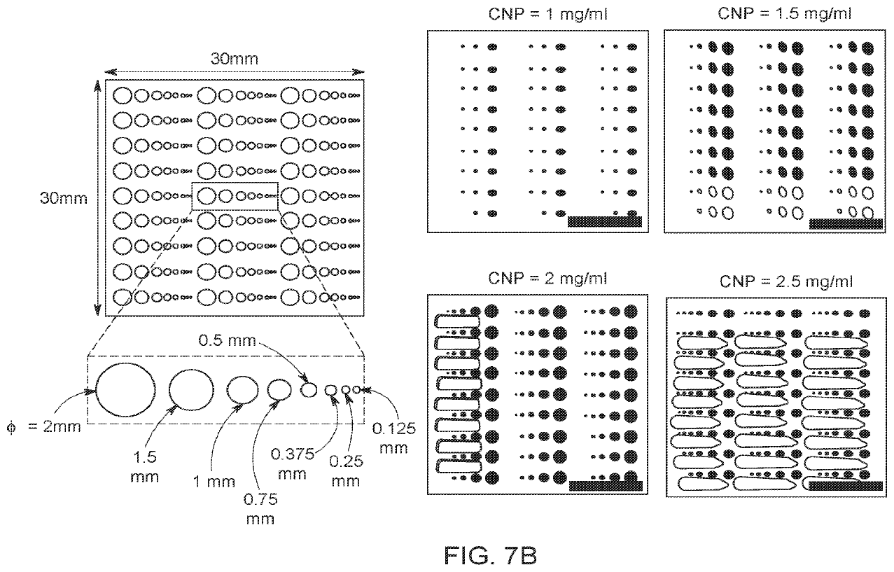

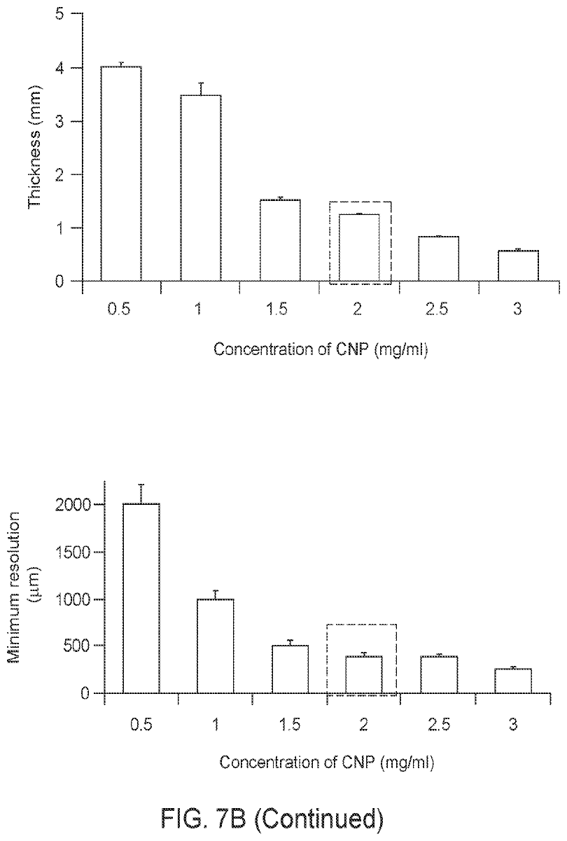

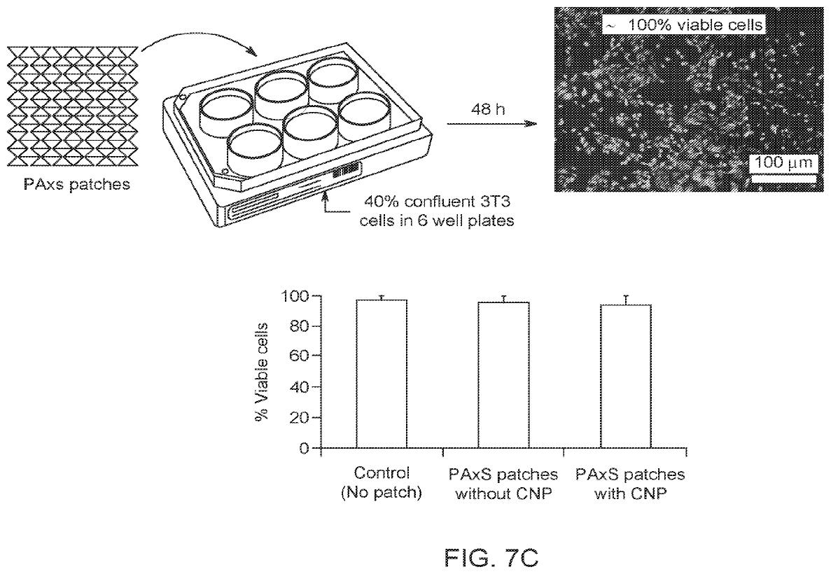

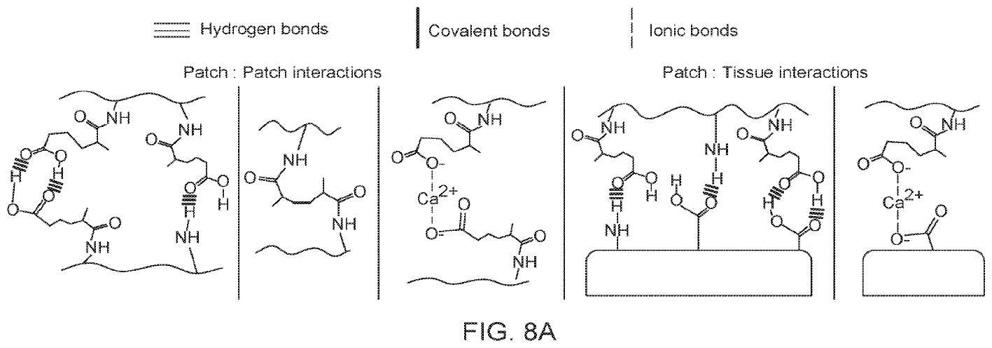

PAxS patches—material composition and patch fabrication. The present patch system, Anisotropic and Auxetic Smart (PAxS) patches, is based on a novel combination of gelatin methacryloyl (GelMA), acrylic acid (ACA), calcium chloride (CaCl₂)), and curcumin nanoparticles (CNPs) to deliver the desired characteristics to conform to dynamic organ mechanics and achieve therapeutic functionality. The patch fabrication procedure, shown in FIG. 7A, is simple, cost effective, rapid, and highly reproducible. The material can easily be formulated using off-the-shelf reagents and is selectively photocrosslinkable, rendering it compatible with digital light projection (DLP), which has specific advantages over other conventional or advanced manufacturing techniques. In DLP, a digital micromirror device is used to project the image of a construct one layer at a time, which allows facile fabrication of highly complex auxetic architectures, which is otherwise a challenging using conventional techniques such as casting or electrospinning. As seen in FIG. 7A PAxS patches are fabricated via a facile single-layer projection-printing of a photoink (GelMA+ACA) contained in a PDMS-coated substrate. After washing and neutralization, calcium chloride is used to enhance patch integrity and adhesion.

Gelatin is chosen as the backbone polymer for the matrix as it is widely studied and has high biocompatibility, possesses integrin binding sites (RGD), and can undergo enzyme-mediated biodegradation. GelMA is prepared through the methacrylation of gelatin and can undergo rapid chain growth polymerization in the presence of a photoinitiator, providing excellent compatibility with high-resolution photocrosslinking. GelMA has become the gold-standard for bioprinting in recent years due to its easy and replicable production and use in light-based biofabrication techniques such as DLP. LAP (lithium-phenyl-2,4,6-trimethylbenzoylphosphinate) photoinitiator (0.03% w/v) is used due to its compatibility with visible UV light (405 nm) crosslinking and has excellent biocompatibility. The addition of ACA did not affect the photocrosslinking of the matrix formulation, and the use of a collimated light beam in DLP allowed patch fabrication as a single layer in under 2 minutes, substantially faster than extrusion printing.

CNPs act as a UV-absorptive and radical quenching agent critical for high-resolution DLP fabrication. The matrix CNP concentration was optimized by determining the print resolution and thickness of template patches with a broad range of feature sizes, illustrated in FIG. 7B, made using different CNP concentrations. Increasing the CNP concentration significantly reduced the minimum achievable feature size (p<0.05) but also reduced patch thickness by affecting the UV penetration depth. Finally, 2 mg/ml CNP was selected, since it achieved a minimum feature size of 375 μm and a consistent patch thickness of ˜1.2 mm, which allowed easy handling of the patches (FIG. 7B).

For any new implantable medical device, the demonstration of biocompatibility is critical. The viability of 3T3 fibroblasts cultured for over 48 h was evaluated in the presence of the patches. In addition to CNP-laden patches, CNP-free patches made using FD&C yellow food dye were also tested, which has previously been shown to demonstrate the radical quenching needed for high-resolution DLP. The presence or absence of CNP had no effect on cell viability (˜100% after 48 h, FIG. 7C).

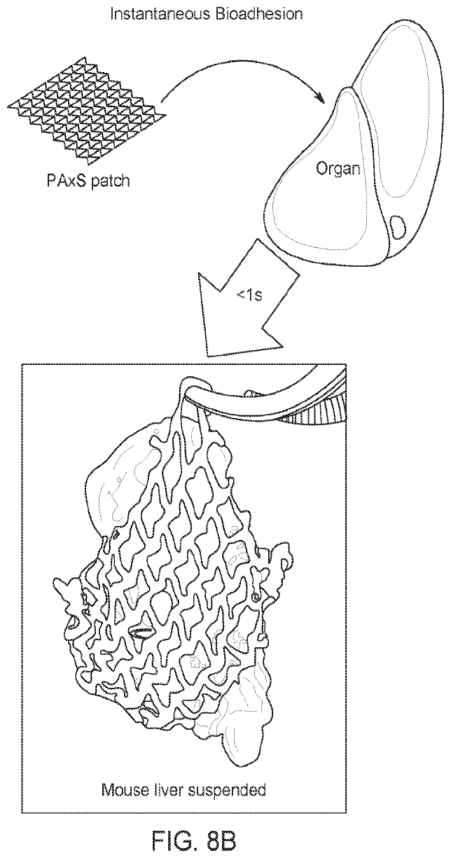

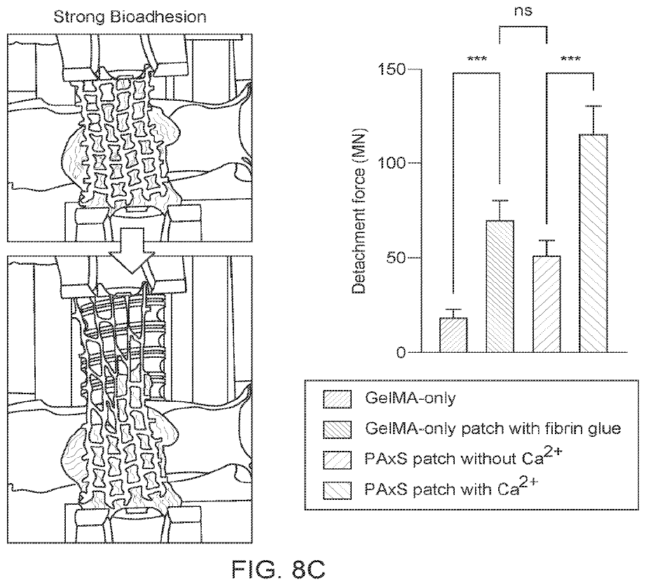

PAxS patches demonstrate instantaneous and strong bioadhesion to wet tissue. A material with strong and instantaneous bioadhesion can substantially reduce the time needed for patch application, thereby improving acceptance from surgeons and minimizing intraoperative harm. GelMA was only negligibly bioadhesive, so acrylic acid and calcium chloride were added to the matrix to impart excellent bioadhesion. Acrylic acid adds carboxylic acid groups to the photocrosslinked matrix, which enhances patch bioadhesion to wet tissue through absorption of interfacial water and hydrogen bond formation (FIG. 8A), while calcium chloride results in ionic crosslinking of patches with the carboxylic acid groups in tissue matrix to further improve bioadhesion, illustrated in FIG. 8A.

Attachment tests were performed on wet organs (FIG. 8B) using a patch formulation containing 80% v/v GelMA solution (5% w/v concentration) and 20% v/v ACA solution treated with 0.5 M CaCl₂) post-fabrication (i.e., PAxS patches with Ca²⁺). The patches demonstrated strong and instantaneous (<1 s) bioadhesion to wet organs and could support the entire weight of a mouse liver upon immediate contact. The forces required to detach the patches attached to mouse livers were further measured (FIG. 8C), and the adhesion force for PAxS patches with Ca²⁺ was almost twice that (p<0.05) of commercially available fibrin glue (TISSEEL, Baxter Healthcare), ˜2.5-times that of groups without CaCl₂) treatment (i.e., PAxS patches without Ca²⁺), and ˜10-times that of GelMA patches. When stretched, patches treated with CaCl₂) stretched and broke but did not detach from the organ, further demonstrating the strength of bioadhesion. Importantly, the PAxS patches with Ca²⁺ not only strongly adhered to tissues but, due to hydrogen bonding and ionic crosslinking within the patches, also imparted the ultra-elasticity needed to conform to various dynamic organs. As seen in FIG. 8C, patches demonstrate strong bioadhesion to mouse liver. The detachment forces for the patch crosslinked in the presence of Ca²⁺ ions are almost two-fold that of patches without Ca²⁺ and higher than those for a GelMA patch attached with an FDA-approved fibrin glue (TISEEL, Baxter). ***denotes p<0.001 (group-wise comparisons carried out via Tukey's honestly significant difference (HSD) post-hoc tests).

PAXS patches are designed to mimic the mechanics of dynamic organs. Dynamic organs have highly variable anisotropy and auxetic or non-auxetic characteristics. For instance, the lungs, myocardium, bladder, stomach, intestines, diaphragm, and the tendons can be considered as dynamic organs, of which the diaphragm and tendons are non-auxetic (positive Poisson's ratio of surface deformation), while other organs are auxetic (negative Poisson's ratio of surface deformation). In addition, intrinsic organ anisotropy is also considered, where the organ stiffness (E) is different in the longitudinal (predominant direction of ECM fiber orientation) and transverse (direction perpendicular to the ECM fibers) directions. Since the void space between the ECM fibers reduces the resistance to deformation in the transverse direction, the longitudinal: transverse stiffness ratio (henceforth referred to as E_{L}:E_{T}) is greater than 1. Except for the lung, which is mostly isotropic (E_{L}/E_{T}˜1), other organs feature anisotropy (E_{L}/E_{T}>1).

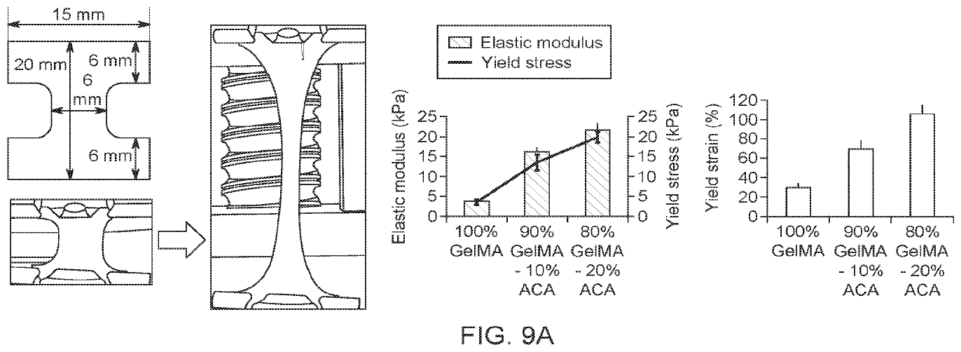

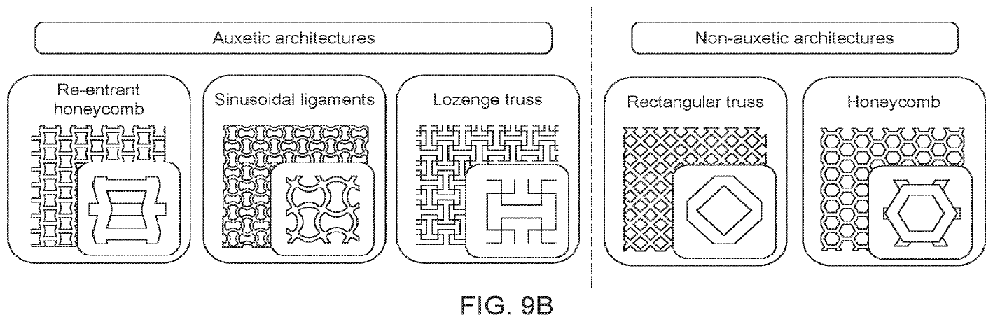

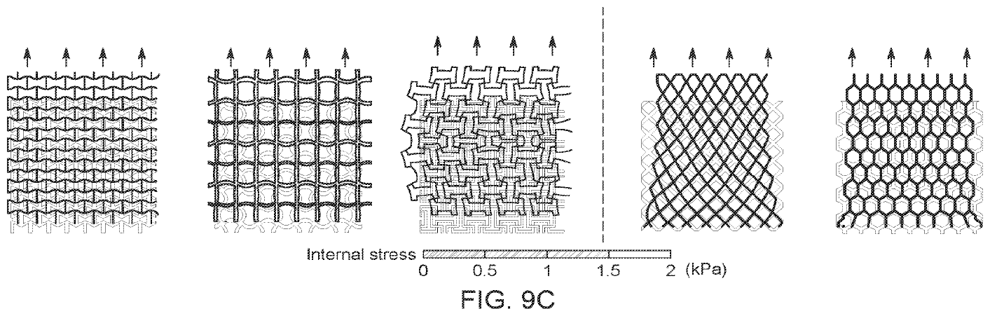

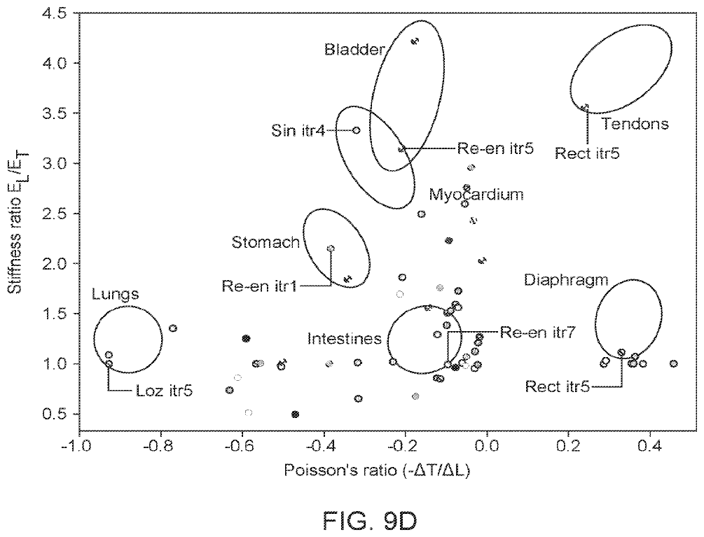

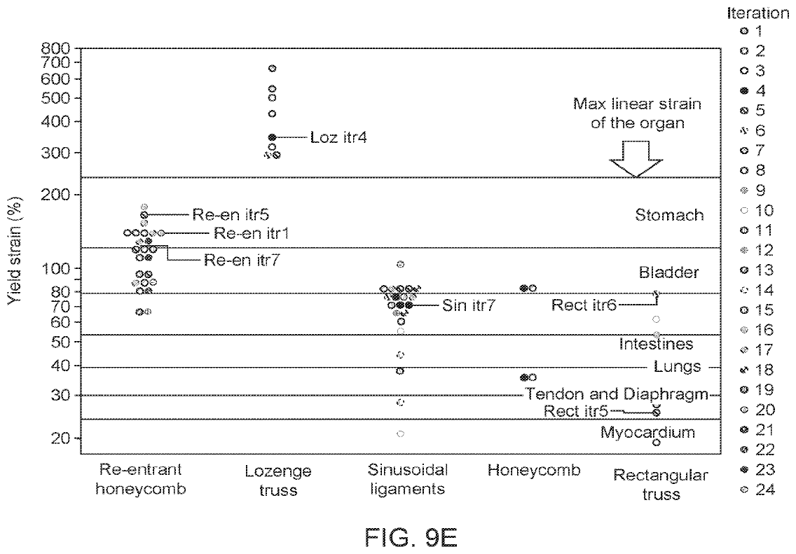

To determine the mechanical characteristics of PAXS patches, structural mechanics computational models were developed to simulate unidirectional (longitudinal or transversal) patch stretching. To establish the material properties of the materials used in the computational model, the tensile strength of dog bone-shaped patches of different formation were empirically measured, as illustrated in FIG. 9A. The 80% v/v GelMA-20% v/v ACA combination demonstrated the highest stretchability (˜110% yield strain) and elastic modulus (˜20 kPa) and was therefore used in the computational models, which were then used to derive patch stiffness in the longitudinal and orthogonal directions sequentially. While many auxetic and non-auxetic architectures have been described in literature, the commonly described re-entrant, sinusoidal ligaments, lozenge truss, rectangular truss, and honeycomb architectures were chosen.

Since patches have hitherto not been designed nor optimized according to specific target organs, a library of 64 designs was created demonstrating a broad range of auxetic, non-auxetic, and anisotropic properties by changing the width (w), height (h), radius (r), inclination angle (ia), and thickness (t), while keeping the overall patch size at 30×30 mm² (FIG. 9B). As shown in FIG. 9C, computational outputs depicting internal stresses within the selected iterations of the geometries of auxetic (negative Poisson's ratio) and non-auxetic (positive Poisson's ratio) architectures using the 80% GelMA-20% ACA formulation. Note that the auxetic patches expand laterally when stretched, while non-auxetic patches contract laterally when stretched. Using this systematic approach, the patch stiffnesses and Poisson's ratios were determined in the longitudinal and transverse directions, as summarized in FIG. 9D, to map the different patch architectures to the dynamic loading of different organs. As seen in FIG. 9D, plots of the patch stiffness ratios, Poisson's ratios, and yield strains and overlap with the properties of individual organs. The yield strain plot lists the maximum linear strain that the organ undergoes in its normal physiological cycle. The patch fit to each dynamic organ was decided based on both the stiffness curves and Poisson's ratios. It was further validated whether these patch architectures could withstand the different deformations of different organs by plotting the yield strains of these patches, FIG. 9E, which clearly shows that the yield strains of the patch formulations for different organs were always in excess of the maximum organ deformation such that the patches would maintain their elasticity throughout repetitive cycles of organ deformation. As seen the yield strain is always greater than that of the normal physiological strain of the organ.

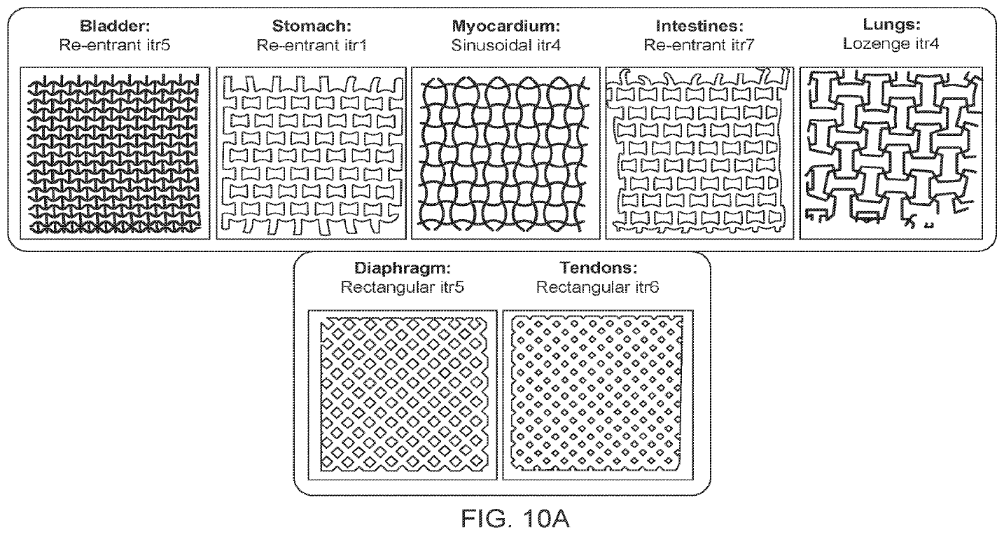

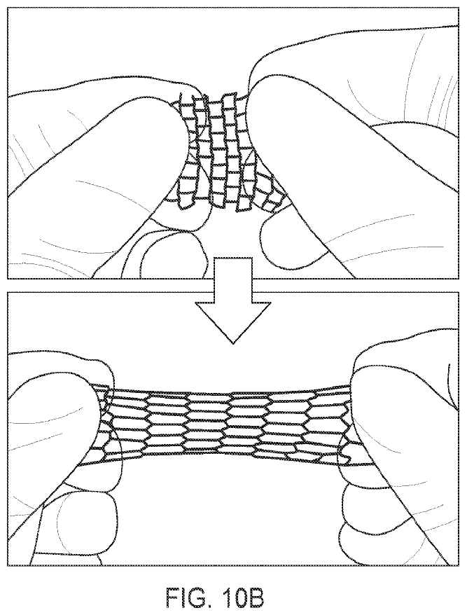

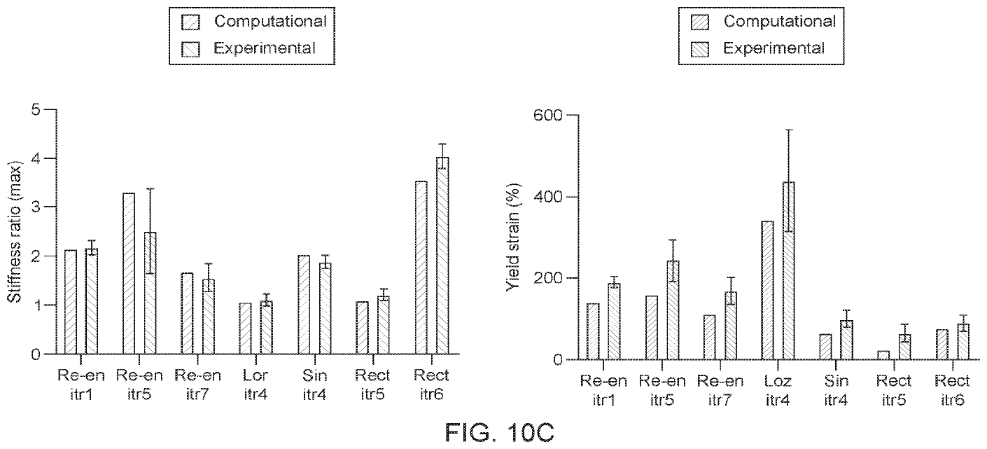

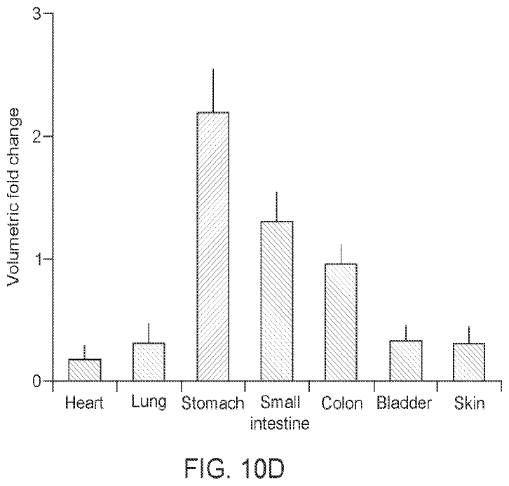

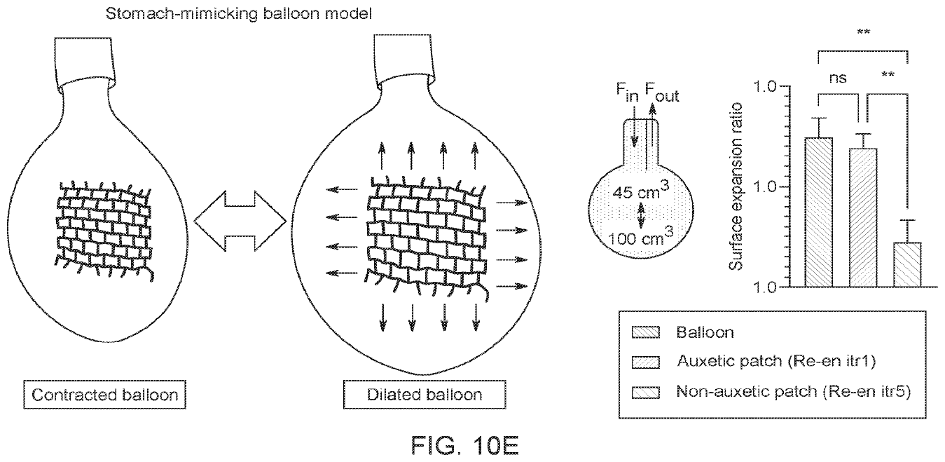

Collectively considering the Ashby plots for organ mechanics in FIG. 9D and the yield strains in FIG. 9E allowed for the finalization of a patch architecture and its iterations for each organ, as illustrated in FIG. 10A. These patches were all ultra-stretchable (FIG. 10B, showing ultra-elasticity and/or stretchability). Tensile testing was performed on each of the selected patch geometries for different organs to validate the computational predictions, which revealed a close correlation between the computational estimates and the experimental outcomes (FIG. 10C, showing tensile testing of the selected patches for the different organs demonstrates a close correlation between the computational and experimental outcomes of the elastic modulus (stiffness) and the yield strains of the patches). The yield strains of the patches were slightly higher experimentally than in silico, which might be because the computational models used perfect geometries with sharp edges, which can give rise to concentrated stress, whereas the sharp edges used experimentally had a fillet radius due to process limitations, which might reduce internal stresses.

Testing was further conducted as to whether the patches could conform to a dynamic organ by determining the change in organ surface area, a direct correlate of the change in patch surface area. Since each organ has a distinct shape that further varies throughout the organ, volumetric fold change of the organ was used as the rational criterion for determining the change in organ surface area (FIG. 10D showing volumetric fold change of different dynamic organs.

The stomach has the highest volumetric fold change compared with other dynamic organs). Of the different organs, the stomach undergoes the highest dilation (up to 2.2-times its original volume) 1, so the stomach was used as the exemplar by developing a mechanized setup to inflate and deflate a stomach-mimicking balloon (FIG. 10E showing Re-en itr 1 patches tested on a dilating balloon model demonstrate adherence to the balloon mechanics and a similar surface expansion to that of the balloon. **represents p<0.01 (group-wise comparisons carried by Tukey's HSD post-hoc tests). The air flow rates inside and outside of the balloon were finely calibrated such that the expansion of the balloon mimicked the surface area change of the stomach and attached the selected stomach-mimicking Re-en itr1 patches onto the balloon model and studying the patch mechanics. Upon increasing the balloon volume to 2.2-times its original volume using cyclical pressurization, auxetic Re-en itr1 patches easily conformed to balloon expansion and contraction and had a similar expansion ratio to that of the auxetic surface of the balloon.

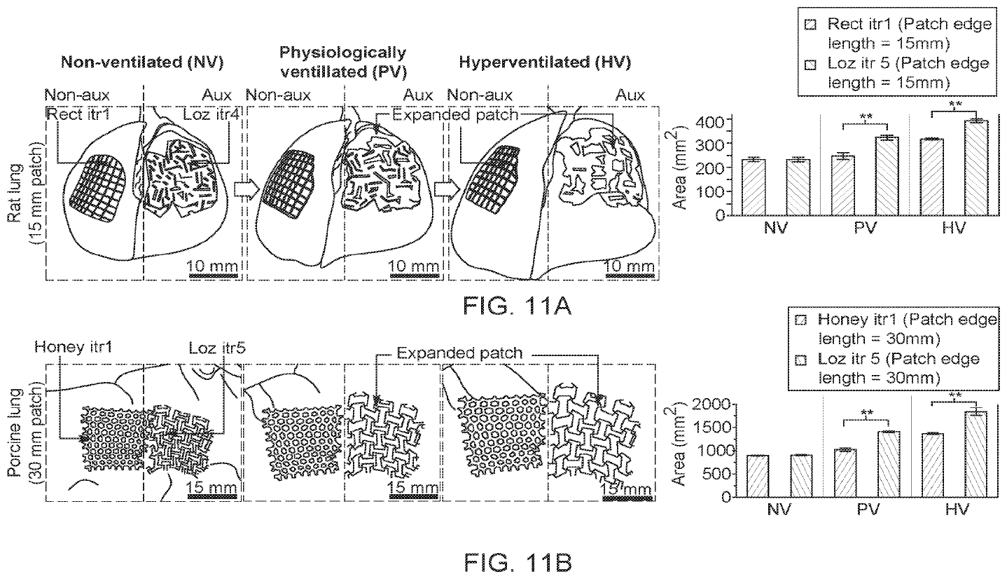

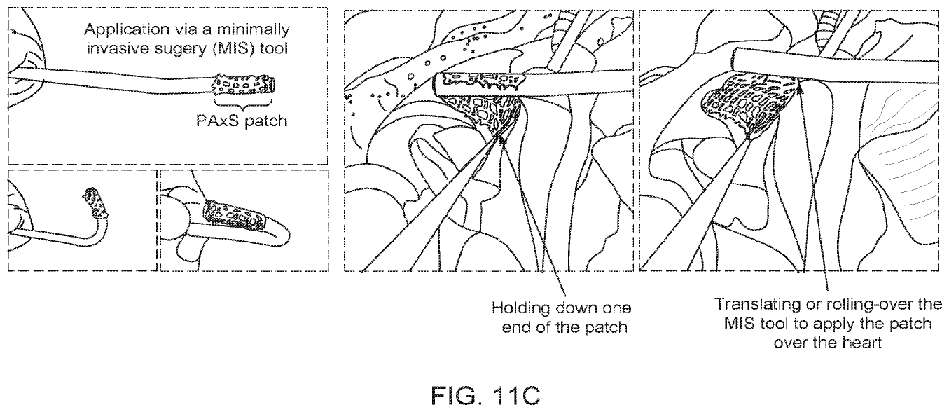

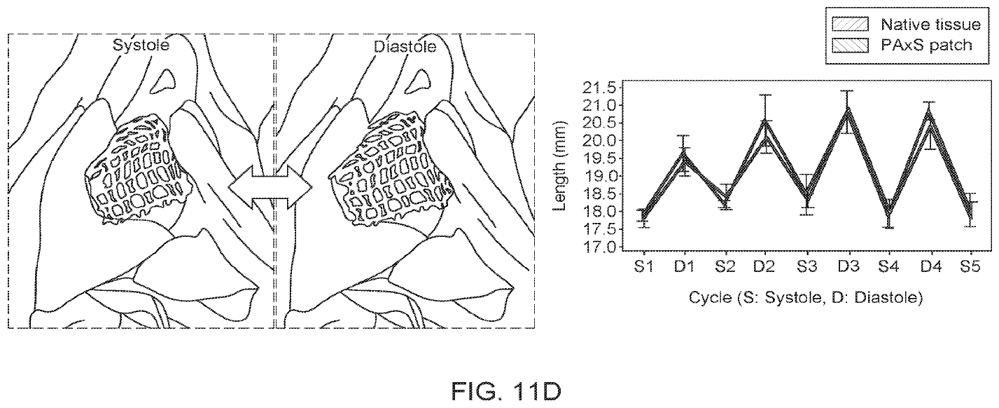

PAxS patches demonstrate conformation to different dynamic organs. An important criterion for any dynamic organ patch is the need to withstand the cyclical stretching and contraction and the resulting fatigue loading after administration. Therefore, ex vivo lung (rodent and porcine) and in vivo heart (rodent) models were used to test patch compliance. For the rodent models, the patches were scaled down by a scaling factor of s=0.5 (i.e., 15×15 mm² patches with proportionally reduced features) and in porcine models a scaling factor s=1.5 was used (i.e., 45×45 mm² patches). Herein, the scaling of the patches will not affect the stiffness and Poisson's ratios of the patches, since all the features are uniformly scaled, and their relative dimensions (e.g., the width and height ratios, etc.) remain the same. Unlike non-auxetic patches, the auxetic patches demonstrated a greater increase in overall surface area during physiological ventilation (PV) and hyperventilation (HV) states (FIG. 11A and FIG. 11B p<0.05) in both the rodent and porcine models. FIG. 11A shows ex vivo implantation of the patches (15) over rat lungs, and comparison of a patch with square holes vs an auxetic patch (Loz itr5). Auxetic patches substantially increased their surface area by increasing the size of the voids, thereby conforming to the lung mechanics. FIG. 11B shows ex vivo implantation of the patches (30×30 mm²) over the right lobe of porcine lungs, where auxetic patches (Loz itr4) better conformed to the lung mechanics compared with non-auxetic (honeycomb) patches.

Next, to demonstrate the compatibility of PAxS patches with minimally-invasive surgery (MIS), the PAxS patches (heart-mimicking Sin itr4) were wrapped around a bronchoscope and applied the patches over the beating heart within an in vivo rodent model. The patches easily complied with the rapid motion of the heart and demonstrated rhythmic stretching and contraction in synchrony with the diastolic and systolic cycles of the heart, illustrated in FIG. 11C. Further, FIG. 11D shows the attached patches conform to the heart beat over several systolic and diastolic cycles. **represents p<0.01 (group-wise comparisons by Tukey's HSD post-hoc tests).

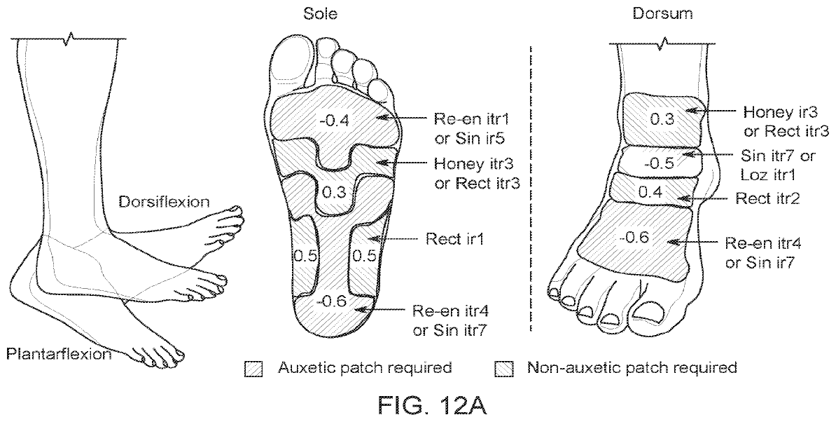

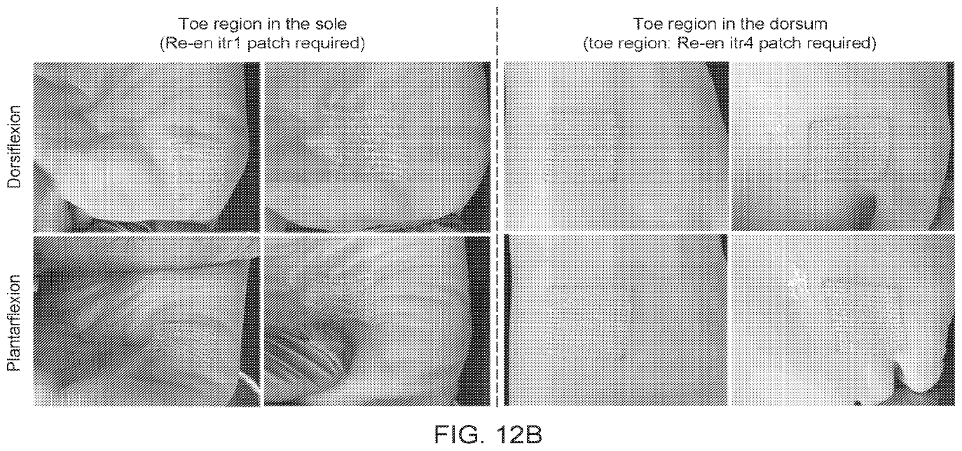

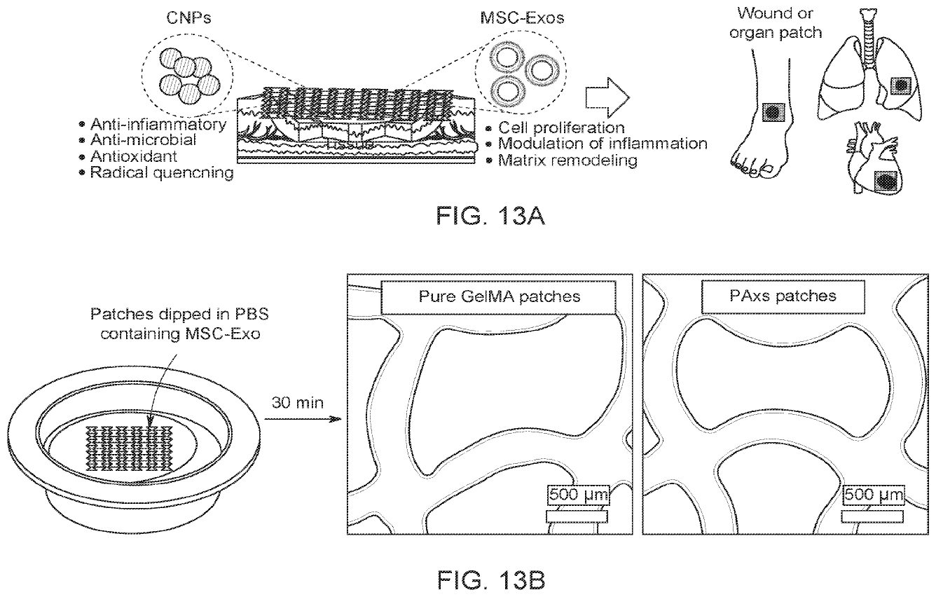

Compared with dynamic organs such as the lung and the heart, the skin undergoes complex deformations during normal physiology. For example, the sole and the dorsum undergo the highest deformation during dorsiflexion and plantarflexion, respectively, and the Poisson's ratio is negative or positive across different regions. Such a large variation in skin mechanics mandates the use of region-specific auxetic or non-auxetic patch architectures. FIG. 12A demonstrates the high variability in the Poisson's ratios in different skin regions, in a synthetic anatomical foot model, from plantarflexion to dorsiflexion of the sole and the dorsum. In some regions, the Poisson's ratio can be close to −0.6 (central region in the heel portion of the sole and toe portion of the dorsum), thus requiring the use of auxetic patches. In contrast, other regions can exhibit positive Poisson's ratios of up to 0.5 (lateral regions of the heel portion of the sole), so non-auxetic patches would be more suitable for long-term administration. FIG. 12B demonstrates conformation tests of the patches in the toe regions of the sole (Re-en itr1 most suitable) and dorsum (Re-en itr4 most suitable) during dorsiflexion and plantarflexion in an anatomical foot model. The patches demonstrated excellent conformation to the movement of the foot, for example, on the sole, Re-en itr1 expanded along both the length and width during dorsiflexion, and contraction along both the length and width during plantarflexion. The patches can conform to the skin dynamics in each case.

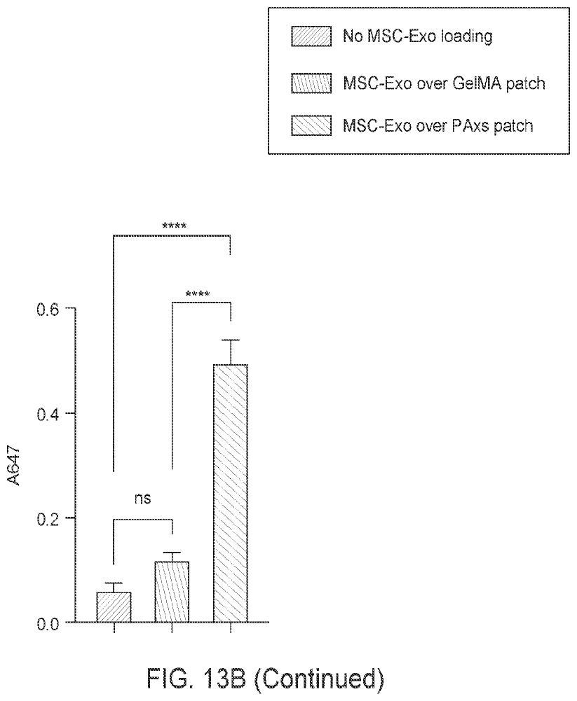

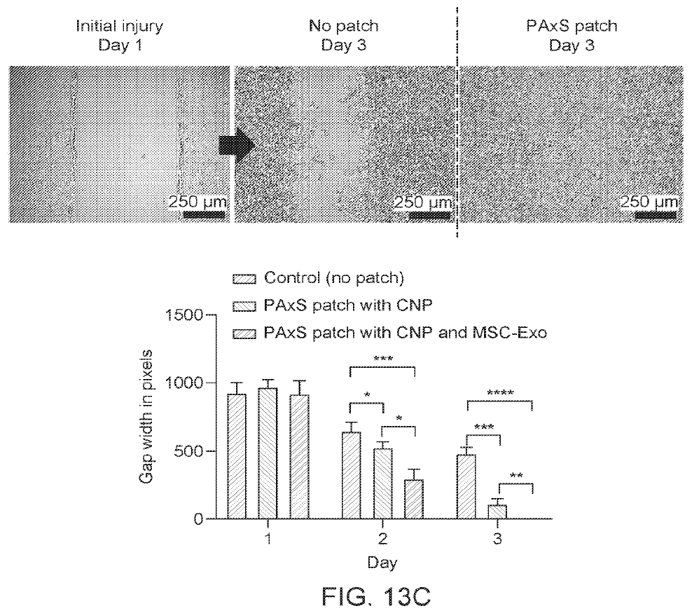

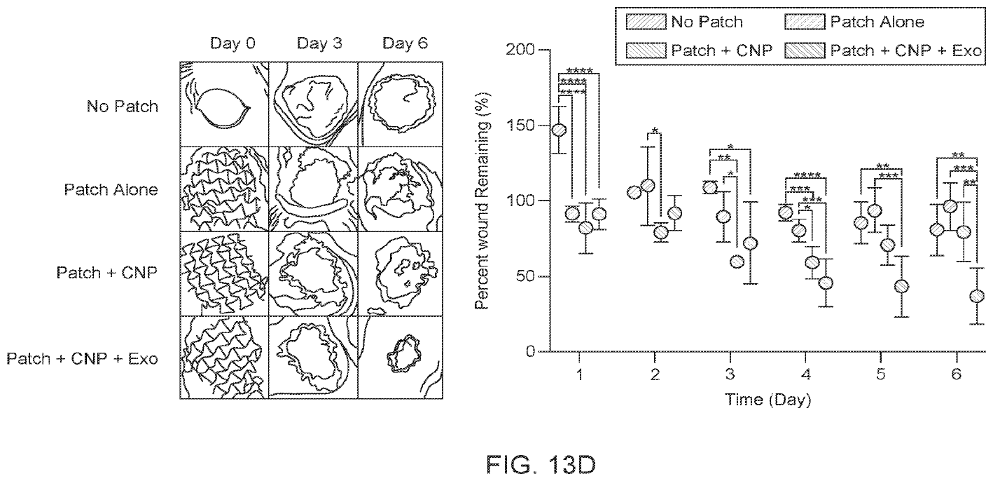

PAxS patches are effective at wound healing. The high conformation of PAXS patches to skin movements should prevent constrained movement after application and patch-induced mechanical stretch of the skin, lending themselves to the personalized treatment of burn wounds and diabetic ulcers with specific patches in different skin regions. The use of PAXS patches for the treatment of skin injuries was investigated using in vitro and in vivo wound-healing models. In this design, CNPs are specifically chosen over other UV absorptive agents (such as FD&C yellow food dye), as CNPs have added therapeutic benefits through their intrinsic anti-inflammatory, anti-microbial, anti-oxidant, and free-radical-scavenging properties. It was further hypothesized that the intrinsic bioadhesiveness of the PAxS patches coated with biological therapeutics can be exploited to further enhance their therapeutic potential. For this, mesenchymal stem cell-derived exosomes (MSC-Exo) were selected as the biological therapeutic, since MSC-Exos have been shown to promote cell-cell interactions, modulate inflammation, and promote cell proliferation, making them an effective treatment for wound healing (FIG. 13A). It was first assessed whether exosomes could be successfully attached to the patches by dipping the patches into a solution containing fluorescently-labeled exosomes (10¹¹ exosomes/ml), followed by quantification. As seen in FIG. 13A, the anti-inflammatory, antimicrobial, and antioxidant properties of CNPs can be combined with pro-proliferative and anti-inflammatory properties of MSC-derived exosomes (MSC-Exos) to improve wound healing. PAxS patches with active bioadhesion clearly outperformed pure GelMA patches (FIG. 13B). After 30 min in the exosome-rich solution, GelMA patches only fluoresced at the edges due to negligible bioadhesiveness, while PAxS patches demonstrated exosome coating across the entire patch surface. As further seen in FIG. 13B, due to their intrinsic bioadhesiveness, PAxS patches allow the attachment of fluorescently-labeled exosomes to their surface. After 1 h of incubation in an exosome-rich solution, the absorbance of the PAXS patches is substantially greater than that of pure GelMA patches.

Next in vitro scratch assays were used to evaluate the effectiveness of PAXS patches for facilitating wound healing. PAxS patches laden with both CNPs and MSC-Exos completely filled the scratch over 48 h, patches laden with only CNP resulted in ˜90% filling of the scratch, while control groups without any patch treatment only filled 40% of the scratches after 48 h (FIG. 13C). As demonstrated in FIG. 13C, in vitro scratch assays with 3T3 fibroblasts demonstrate that patches laden with both CNPs and MSC-Exos clearly outperform patches laden with CNPs and the control groups (no patches). While the control groups only demonstrated ˜40% wound healing after 48 h (days 1 to 3), CNP-laden patches demonstrated up to 90% healing and the MSC-Exos and CNP-laden patches demonstrated complete wound healing.

The wound healing effectiveness of the patches was then assessed (including without CNPs but with food dye as a UV absorptive agent), in a mouse model of cutaneous injury. The patches attached easily to the mouse backs and conformed to the movement of the animal. After 24h of patch treatment, all patch groups demonstrated significant wound healing compared with controls (no patch). After 3 to 4 days, PAxS patches laden with CNPs and PAxS patches with both CNPs and MSC-Exos demonstrated significantly better wound healing compared with controls and animals with patches without CNPs, indicating that the CNPs and MSC-Exos also contribute to short-term wound healing. However, after a week, only PAxS patches with CNPs and MSC-Exos demonstrated significantly increased wound closure compared with other groups. MSC exosomes have been shown to promote cutaneous wound healing through macrophage polarization to the anti-inflammatory M2 phenotype and by promoting collagen secretion and angiogenesis, which likely synergized with CNPs to provide robust wound healing.

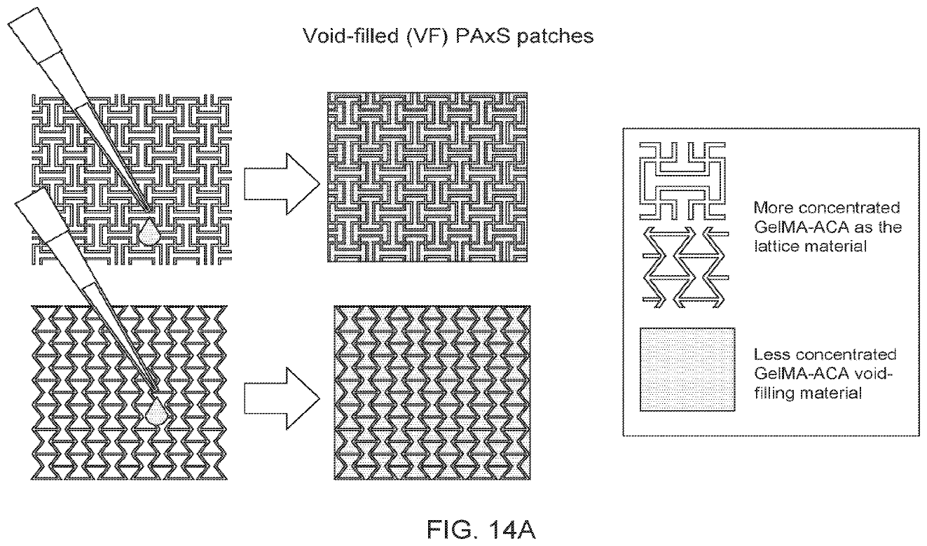

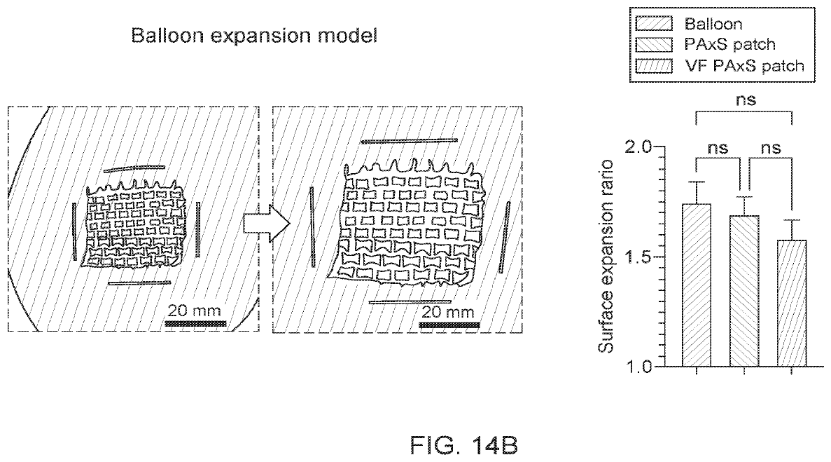

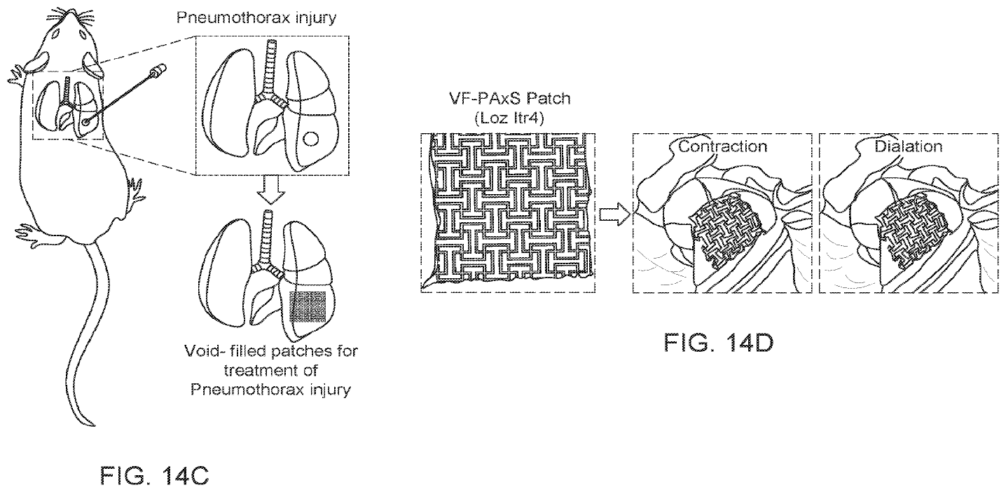

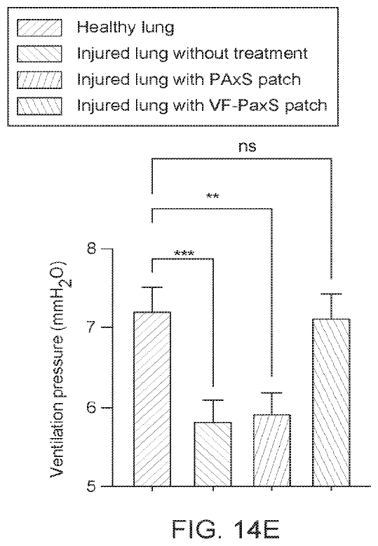

Void-filled PAxS patches are an effective treatment of puncture wounds. While auxetic patches are well suited to many dynamic organ pathologies (e.g., myocardial infarction treatment), they are unsuited for pathologies involving loss of bodily fluids or air (pneumothorax). The PAxS patches were therefore modified using a lower concentration GelMA-ACA mixture to create a first-of-its kind, hole-filling, auxetic patches by filling the voids in the auxetic lattice within the patches (FIG. 14A showing VF-PAxS patches were fabricated by filling the voids of PAXS patches using a reduced concentration of matrix containing GelMA and ACA). Optimization of the concentration of void-filling (VF) material to successfully fill puncture wounds whilst still retaining the auxetic properties was achieved. In this formulation, the 80% v/v GelMA-20% v/v ACA mixture used for the patch lattices was further diluted to 50, 60, 70, 80, and 90% of its original concentration by mixing with PBS and application to the patch voids followed by UV exposure. Void-filling material diluted to 80% of its original concentration was auxetic. FIG. 14B shows the balloon dilation measurements of the optimized VF-PAxS patches) and retained its integrity on stretching. As shown in FIG. 14B, PAxS patches synthesized using a 20% lower concentration of material than that used for the lattices (80% v/v GelMA and 20% v/v ACA solutions) demonstrated auxetic properties on balloon deformation without opening matrix pores. This novel combination of void-filling materials into patches could be an effective strategy for the treatment of pulmonary air leakage, shown in FIG. 14C, illustrating the concept of using VF-PAxS patches for the treatment of pulmonary air leakage in an in vivo rat model.

The use of VF-PAxS patches for pulmonary air leakage in an in vivo rat model was also investigated. Pulmonary air leakage was induced in SD rats by open chest surgery and puncturing the lungs with an 18G needle. The instantaneous and strong bioadhesiveness of the patches allowed patch placement while the lung underwent normal physiological motion (FIG. 14D, showing lung-mimicking VF-PAxS patch and its placement over rat lung) a clear advantage over conventional patches requiring the lung to be static for application. The change in tidal ventilation pressure in injured lungs without treatment or after treatment with simple PAxS patches (without void-filling material) and VF-PAxS patches was then compared. Healthy lungs demonstrated a ventilation pressure of 7.2 mmH₂O under dilation, which was reduced to 5.8 mmH₂O in injured lungs (FIG. 14E). As expected, there was no improvement in lung function in the simple PAxS patch group, but VF-PAxS patches fully restored lung function (FIG. 14E, illustrating that after inducing pulmonary injury, the tidal ventilation pressure during dilation of the lung reduced from 7.2 to 5.8 mmH₂O. VF-PAxS patches fully restored the ventilation pressure to physiological levels, while simple PAxS patches were unable to improve the lung function due to air leak through the empty voids within the patch lattices).

As will be appreciated, there has been a steady rise in the development and use of therapeutic patches over the last few decades for a wide range of applications including myocardial infarction, chronic wounds, organ hemorrhage, and cancer. However, most of these patches do not readily adhere to wet and dry tissues and do not consider the complex mechanics and volumetric changes of dynamic organs caused by their auxetic and anisotropic properties. Here, a novel PAxS patch composition and design is provided that addresses the limitations of conventional patches. PAxS patches in accordance with the present technology display the following unique properties: 1. high resolution printability (optimized CNP concentration (2 mg/ml) in conjunction within DLP allows up to 375 μm print resolution); 2. ultraelasticity (stretchable up to 400% its length without breaking); 3. instantaneous bioadhesion to dry and wet tissue (strong bonds form in <1 s over wet tissues); 4. strong bioadhesion (detachment forces are higher than patches applied via commercial fibrin glue); 5. wound healing capability in vivo (through the controlled delivery of CNPs and conjugated MSC-Exos); and 6. possible synthesis with void filling to heal puncture wounds.

Curcumin nanoparticles provided an excellent solution to act as both a therapeutic and a UV absorptive agent. The high (375 μm) resolution derived through the optimized CNP concentration allowed the manufacture of patches that closely mimicked their intended designs, and closely recapitulated the simulated mechanical properties in tensile tests (see FIG. 10). Resolution could be further improved by reducing the voxel size of the incident light beam. While the current system used chain-growth polymerization of the GelMA matrix in the presence of LAP, future research could use step-growth polymerization using thiol-ene photoclick chemistry to allow for both higher resolution fabrication and quicker fabrication times. For other biomedical applications, patches without CNPs can also be fabricated using food dyes as the absorptive agent. The GelMA-ACA combination composition is simple enough to easily replicate and fabricate and can be linearly scaled-up for large-scale patch fabrication. Since the patches can be manufactured in a single layer, custom masks can also be used with UV lamps to fabricate the patches, negating the need for complex projection systems in 3D printers and further reducing the cost of fabrication.

In addition to the use of simple materials for fabrication, the patches are also easy to use due to instantaneous and strong adhesion to wet tissue. These properties also improve the clinical translation potential of the technology, since organs do not need to be immobilized for several seconds to minutes for their application, a current requirement for some patches reliant on the formation of amide bonds for crosslinking. Other mussel-inspired formulations relying on hydrogen bonding and free radical polymerization have demonstrated instantaneous adhesion, but these formulations rely on dopa and its catecholic groups, which frequently undergo oxidation in neutral and basic conditions and require a fine redox balance to maintain tissue adhesion. This is difficult to achieve and severely compromises their adhesion and limit their practical applications in medicine. Further, this rational design framework for organ-specific soft anisotropic-auxetic patches ensures fatigue resistance over long-term administration while preventing undue stress on the organ.

These wound healing experiments strongly suggest that a dual-therapeutic patch system containing MSC-Exos and CNPs is highly effective for promoting wound healing. Notably, PAxS patches demonstrated gradual dissolution into the skin after a week (FIG. 13D), which could be desirable since the patches would not need to be surgically removed after the wound has healed. This property may be less advantageous for deep-scar wounds or chronic injury such as myocardial infarcts. Since the photocrosslinking density plays a major role controlling the rate of disintegration, a higher density of covalent crosslinks within the patches might prolong the time to dissolution and could be fine-tuned for different applications by increasing the degree of methacrylation and concentration of GelMA or the content of photoinitiator and UV exposure. As shown in FIG. 13D, PAxS patches laden with CNPs and MSC-Exos demonstrated significantly greater wound closure compared with other treatment groups after a week of administration over cutaneous wounds in mice. The effect of CNPs on wound healing was also evident in the short-term, with CNP-laden patches demonstrating better wound healing compared with patches without CNPs until day 4. * indicates p<0.05, ** indicates p<0.01, ***indicates p<0.001, and ****indicates p<0.0001 (group-wise comparisons by Tukey's HSD post-hoc tests).

Only a few patches have been clinically approved, highlighting the challenges to clinical effectiveness and translation. Other adhesive materials have also been investigated with promising strong and instantaneous adhesion and ultra-elasticity, but the materials lack rationally designed anisotropic and auxetic architecture, ultra-elasticity, instant bioadhesion, and compatibility with photoprojection. Regardless of the material used, the rational design framework demonstrated here can be used to design the anisotropic and auxetic patches to conform to specific organs, thus improving long-term integration of patches within hosts. With these VF-PAxS patches, it has been demonstrated how this rational design framework can be further expanded to also include the treatment of dynamic wounds such as pulmonary air leakage. Accordingly, this technology provides demonstration of this combined scheme, which allowed retention of auxetic properties whilst also preventing gas leakage from the lung. Accordingly, aspects of the present technology provide a versatile platform for easy adaptation with different biomaterials and application to a wide range of applications including battlefield injuries and other diseases.

Patch materials synthesis. GelMA was prepared using existing protocols for the controlled methacrylation of gelatin. Briefly, 5 g of gelatin (Bloom 300, porcine derived, Sigma Aldrich, St. Louis, MO) was dissolved at 10% w/v in 0.25 M carbonate-bicarbonate buffer containing 2% w/v of sodium bicarbonate and 0.12% w/v of sodium carbonate in deionized water. The gelatin solution was kept at 50° C. until achieving a clear solution. Next, 159 μl of methacrylic anhydride (MA) was added and the reaction allowed to run for 60 min at 50° C. The reaction was stopped, and excess MA removed, by the addition 100 ml of 1:1 mix of pure ethanol and acetone. The degree of methacrylation was 40%, as determined by ¹H NMR. Acrylic acid was used as purchased (79-10-7, Sigma Aldrich). CNPs were prepared using established methods. Briefly, curcumin (Cur) powder (C1386, Millipore Sigma) was dissolved in tetrahydrofuran (THF) solution at 25 mg/mL. 50 μL of the Cur-THF solution was rapidly injected into 450 μL of deionized water with vigorous stirring at 1400 rpm to aggregate as nanoparticles. The CNP suspension was air-dried to remove the organic solvent and lyophilized. The resultant CNPs were stored at −20° C. until further use. To prepare the material for the patches, 10% w/v GelMA at 37° C. and acrylic acid (79-10-7, Millipore Sigma) were mixed at the desired ratio (80%-20% or 90%-10% v/v), followed by the addition of 0.03% w/v LAP photoinitiator and the desired amount of CNPs (1-2.5 mg/ml) to formulate the photoink.

Patch printing and postprocessing. The material was cooled to room temperature (24° C.), and 750 μl was added to a rectangular enclosure (50×50 mm²) attached onto a PDMS-coated petri dish and uniformly smeared across the enclosure using a pipette tip. Acrylic acid prevented the thermo-reversible crosslinking of GelMA, which is otherwise a native property of GelMA hydrogels. The petri dish was then placed above a digital light projection system (LumenX, Cellink AB) and projection printed using 405 nm UV light at 20 mW/cm² for 2 min. Patches were then neutralized and further crosslinked by dipping into a solution containing 0.5M CaCl₂ and 0.05M NaOH in deionized (DI) water. Before any further experiments, the patches were gently washed with DI water.

Tensile testing. The tensile properties of the patches were determined using an in-house setup consisting of a linear stage actuator (101-80-124, SainSmart) as the stretching mechanism, and a 5 kg load cell (TAL220B, Sparkfun) and amplifier (HX711, Sparkfun) as the stationary anchor. The linear actuator was controlled via a stepper driver (101-60-197, SainSmart) and motion controller (101-60-199, SainSmart). The ends of the patch were clamped in-between the linear actuator and the load cell, and the patches stretched 0.125 mm/s similar to other studies. The load cell was connected to an Arduino Uno and provided the stress vs. strain curve to determine the mechanical properties.

Bioadhesion measurements. Patch bioadhesion was measured over explanted mouse livers. Briefly, the mouse livers were gently dabbed using a blotting paper to remove excess blood followed by placement over the stationary load cell of the tensile testing apparatus. Next, patches were placed on the mouse liver. For GelMA patches administered using fibrin glue, the fibrin sealant was first prepared as per the manufacturer's guidelines (TISSEEL, Baxter). GelMA patches were placed on the mouse livers, followed by placement of 50 μl each of the fibrinogen prepolymer solution and the thrombin crosslinker solutions to attach the patches to the mouse liver. All patches were administered such that one end of the patches hanged over the edge of the liver by 5 mm, which was attached to the linear translation stage of the tensile testing apparatus. The patches were stretched at 0.125 mm/s and the highest force generated during the stretching procedure was noted as the detachment force.

Evaluation of patch biocompatibility in vitro. NIH 3T3 fibroblasts (CRL-1658, ATCC) were cultured in Dulbecco's modified Eagle's medium (DMEM) and 10% fetal bovine serum and 1% penicillin-streptomycin in six-well plates (2 ml of medium per well) until 40% confluency. The patches were then added into the wells and the viability assessed after 2 days using the Live/Dead™ assay (L3224, ThermoFisher Scientific).

Patch designs. Solidworks (Dassault Systems, Vélizy-villacoublay, France) was used to prepare the patch designs. The inset images (Error! Reference source not found.A) constituted the repetitive elements in the linear array of the lattice. The dimensional parameters (h, w, r, t, the, and ia) were defined as global variables, so that they could be easily altered to change the overall patch design. The overall size of the patches, irrespective of the dimensionality of the lattice elements, was kept at 30×30×1 mm³.

Computational modeling of the patches. The 3D patch geometry was imported into the structural mechanics module of COMSOL Multiphysics (COMSOL Inc.). A fixed boundary condition was applied on one edge of the patch (either the longitudinal (L, assumed along x-axis) or transverse (T, assumed along y-axis) direction) and a linear displacement of δ=10% strain applied on the opposite edge. A mesh density of 1/10th of the minimum element size (0.25 mm) was selected as previously. The stiffness of the patch (E) was determined from the strain energy (Us), strain (δ_{L} or δ_{T}=10%), and volume of the patch (V_{P}) as per E=δ²V_{P}/2Us. For some patches, this stiffness was different when stretched longitudinally (along x-axis), than when stretched transversally (along y-axis), thereby making the stiffness ratio smaller or larger than 1. The Poisson's ratio (vₚ) was determined from the deformation transverse deformation when stretched longitudinally, hence vₚ=δ_{T}/δ_{L}. The yield strain (δ_{Yield}) of the material was calculated at the stretch when the maximum internal stress (von mises=σᵢ) within the patches exceeded the yield strength (σₘₐₓ) of the bulk material: δ_{Yield}=σᵢ/σₘₐₓ.

Balloon model demonstrating patch compliance to organ mechanics. The balloon model consisted of a programmable control of a pneumatic valve using a linear actuator capable. An air flow meter was used to calibrate the air flow (VFA-26-BV, Dwyer Instruments). An open cylindrical tubing attached to the inlet of the balloon allowed the input air to exit the balloon. To measure the change in the surface area of the patches compared with that of the balloon, point marks were placed on the portion of the balloon encompassing the patches and around the patches. The relative expansion of the patch and the balloon was measured by calculating the distance between the opposite edges of the patches, and the opposite points placed along the balloon, in ImageJ. The product of the measured length (L) and width (W) was the surface area. The expansion ratio was the ratio between the change in surface area of the patches (L_{P}×W_{P}) to the change in surface area of the square encapsulated by the points on the balloon (L_{B}×W_{B}).

Ex vivo evaluation of patch compliance. Rat lungs were excised from male Sprague Dawley (SD) rats (˜300 g average weight, 12 weeks old) after euthanasia. Porcine lungs (cold flushed post euthanasia) were obtained from North Carolina State University School of Veterinary Medicine. Patches were attached to the lungs using forceps. The lungs were then ventilated using a rodent ventilator and a porcine ventilator, respectively. The ventilation volumes were 7.5 ml/kg (physiological ventilation) and 12.5 ml/kg (hyperventilation) for rat lungs, and patch motion was recorded as a video. The patch size was determined as for the balloon model.

In vivo evaluation of patch compliance. An intraperitoneal injection of ketamine/xylazine was used to anesthetize the SD rats and the rats were kept unconscious using ventilation at a volume of 7.5 ml/kg (typical for rodent studies⁸⁵) with isoflurane and oxygen (after tracheotomy). The lungs and the heart were exposed via sternotomy. The patches were wound around a bronchoscope and attached onto the heart after holding on one end near the bottom of the left ventricle. Patch motion was recorded as a video. The changes in patch length were determined using analysis of different frames of the captured video in ImageJ.

In vitro healing evaluation using scratch assays. Methods based on previous work were used for the scratch assay. Briefly, 3T3 cell cultures within six-well plates were allowed to reach 100% confluency, and were then scraped using a 23G needle to create ˜650 μm (wide) and 5 mm (long) scratches through the cells. The cells were then allowed to proliferate through the scratch without any treatment (control), or in the presence of patches (placed within the wells after creation of the scratch) containing only CNPs or both CNPs and MSC-Exos. The width of the scratch was determined after 24 and 48 h using analysis of brightfield images in ImageJ.

In Vivo Murine Wound Healing Model. Female, 6-8 week old C57BL/6J were purchased from Jackson Laboratory. The animal studies were approved and carried out in compliance with the Institutional Animal Care and Use Committee standards. The mice were housed individually with 12-h light-dark cycles. For wounding, mice were anesthetized using gaseous isoflurane and received a subcutaneous injection of 0.05 mg/kg buprenorphine. Hair was removed from the dorsal region of the mouse using clippers and a depilatory cream, and the skin was prepared for surgery using betadine and 70% ethanol. A sterile 6 mm biopsy punch was used to outline a circular pattern between the shoulders. Forceps were used to lift the skin, and surgical scissors were used to create a full-thickness wound on each mouse. Mice were randomly assigned into four treatment groups of 5 mice each and treated with an unloaded patch, a curcumin nanoparticle loaded patch, a curcumin nanoparticle plus mesenchymal stem cell exosome loaded patch, or no patch. Patches were adhered to the wound site immediately following wounding. After the initial treatment, wounds were covered with a Band-Aid to facilitate patch adherence and prevent patch removal by the mice in the first 24 hours post-wounding and treatment. Each day, all wounds were measured in perpendicular directions using calipers for wound area calculations, and wounds were imaged. On day 6 post-wounding, mice were sacrificed and residual wounds were harvested and stored at −80° C.

MSC-exosome synthesis, fluorescent labeling, and conjugation over the patches. The serum for the exosome-free medium was prepared by centrifugation of fetal bovine serum (FBS, MilliporeSigma) at 100,000 rcf in an ultracentrifuge for 12 hours, followed by extracting the supernatant. This serum was then mixed at 10% concentration in Dulbecco's modified Eagle Medium (DMEM, Millipore Sigma). MSCs (passage 2) were first cultured in 10% FBS and 90% DMEM until reaching 70% confluency in T-75 flasks. Then, the cells were washed three times with PBS to remove excess serum, and the medium was replaced with exosome-free media. The medium containing MSC-exosomes was collected after 48 h and the exosomes concentrated via a sequential centrifugation, re-suspended in PBS, and quantified by nanoparticle tracking analysis (NTA). To fluorescently label the exosomes, an NHS ester fluorophore (Dylight650, ThermoFisher Scientific) was added at a concentration of 0.1 mg/10¹¹ exosomes, followed by incubation at 37° C. The excess fluorophore was then removed using exosome spin columns (molecular weight cutoff 3 kDa, ThermoFisher). Exosomes were then reconstituted in PBS at a concentration of 10¹¹ exosomes/ml. On each patch (air dried for 5 min), 100 μl of the fluorescent-exosomes suspension was added to allow the exosomes to conjugate for 30 min at 24° C. The patches were then washed three times with PBS to remove excess exosomes. Patches were then cut into 4 sections and dipped in 24-well plates containing 250 μl of PBS, and the absorbance was measured at 647 nm using a plate reader. For confocal imaging (Fluoview, Olympus), the 647 nm laser was used to visualize the patch samples in PBS.

In vivo pulmonary air leakage treatment with composite PAxS patches. SD rats were prepared as for the in vivo compliance studies (physiological ventilation volume of 7.5 ml/kg was used). The tidal ventilation pressure in the airway was continuously monitored with a pressure transducer (Transpac IV, ICU Medical Inc). Pulmonary air leakage was induced in the right lower lobe of the lung by inserting an 18G needle up to 1 cm deep in the lung tissue. Patches were placed over the injury site with forceps. Any changes in ventilation pressure were noted throughout the procedure.

Statistical analysis. All experimental data are presented as mean+standard deviation. For in vitro, ex vivo, and live animal models, a sample size of n=3 was used. Statistical tests were performed in GraphPad PRISM (GraphPad Software, La Jolla, CA). One-way ANOVA was used for the bioadhesion measurements (FIG. 8C), balloon experiments (FIG. 10E and FIG. 14B), loading of MSC-Exos over PAxS patches (FIG. 13B), and pulmonary air leakage treatment experiments (FIG. 14F). Two-way ANOVA was used to assess patch conformation to ex vivo lungs (FIG. 11A and FIG. 11B), and in vitro and in vivo wound healing studies (FIG. 13C and FIG. 13D). Tukey's HSD was used for the post-hoc analysis.

### Example 3—Adhesive and Ultra-elastic Patches for Wound Healing

According to some aspects of the present technology, stretchable and adhesive patches are provided, in some instances referred to as highly-stretchable and/or super-stick (adhesive) patches. In some instances, these patches can incorporate compositions comprising GelMA, acrylic acid (ACA), curcumin, and calcium chloride (CaCl₂). As will be appreciated, the resulting material of the addition of curcumin nanoparticles, GelMA+LAP (photoinitiator), ACA and CaCl₂) and further formation into a patch currently is not known in literature.

After establishing the effectiveness of the MSC-Exo-laden PAxS patches in bridging the scratches in vitro, the effectiveness in wound healing in a mouse model of cutaneous injury was assessed. Herein, in addition to patches containing CNP and CNP+Exos, patches without CNPs were investigated, which were made by adding a food dye as UV absorptive agent instead of the CNPs. The patches could be easily attached to the backs of the mice and demonstrated conformation to the movement of the animal. After 1 day of inducing the wound and applying the treatments, all patch groups demonstrated significant wound healing compared to the control (no patch). After 3 to 4 days, the PAxS patches laden with CNPs and PAxS patches with both CNPs and MSC-Exos demonstrated significantly high wound healing compared to the control and the groups containing the patches without CNPs, indicating stand-alone effect of CNPs and MSC-Exos in contributing to short-term wound healing. However, over a week-long administration, only the PAxS patches with CNPs and MSC-Exos demonstrated significantly increased wound closure compared to other groups. The CNP-laden patches not demonstrating higher healing compared to the groups without CNP after week could be attributed to the dissolution of the patches by the end of the week. This could have affected the therapeutic function of CNPs by limiting their availability. MSC-exosomes, on the other hand have been shown to promote cutaneous wound healing through macrophage polarization to M2 phenotype, and promoting collagen secretion and angiogenesis, which likely compounded the short-time effect of the CNPs to provide robust wound healing. These results indicate that for a longer-term administration, MSC-Exo-laden patches are more effective at wound healing.

Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below. Certain features and subcombinations are of utility and can be employed without reference to other features and subcombinations and are contemplated within the scope of the claims.

1. A patch for organ or tissue application comprising:
a biocompatible polymeric material having an auxetic architecture matching a stiffness ratio and Poisson's ratio of an organ or tissue to which the patch is applied.

2. The patch of claim 1, wherein the auxetic architecture is anisotropic.

3. The patch of claim 1, wherein the biocompatible polymeric material comprises a hydrogel.

4. The patch of claim 1, wherein the biocompatible polymeric material comprises multiple hydrogel layers.

5. The patch of claim 4, wherein the multiple layers have differing hydrogel materials.

6. The patch of claim 1, wherein the stiffness ratio and Poisson's ratio remain unchanged regardless of scaling factor.

7. The patch of claim 1, wherein the auxetic architecture comprises apertures of repeating shape.

8. The patch of claim 1, wherein the auxetic architecture comprises patterned apertures.

9. The patch of claim 7, wherein auxetic architecture comprises lattice structures.

10. The patch of claim 7, wherein the apertures are filled with material blocking or inhibiting passage of liquid and/or gas.

11. The patch of claim 10, wherein Young's modulus of the filler material is less than the biocompatible polymeric material.

12. The patch of claim 7, wherein the apertures have a shape selected from the group consisting of re-entrant honeycomb, chiral truss, lozenge truss, orthogonal oval voids, arrowheads, pinwheels, sinusoidal ligaments, and orthogonal truss.

13. The patch of claim 7, wherein the apertures are filled with one or more therapeutic agents.

14. The patch of claim 13, wherein the one or more therapeutics agents comprises a pharmaceutical, biologic, nucleic acid, protein, or cellular species.

15. The patch of claim 14, wherein the one or more therapeutic agents are operable for wound healing.

16. The patch of claim 13, wherein the one or more therapeutic agents are contained in exosomes.

17. The patch of claim 1, comprising one or more structural features having size of 100 μm to 500 μm.

18. The patch of claim 1, wherein the biocompatible polymeric material further comprises one or more UV-absorbers.

19. The patch of claim 1, wherein surfaces of the patch are populated with moieties operable to form hydrogen bonds and/or ionic bonds with tissue surfaces.

20. The patch of claim 19, wherein the moieties comprise carboxyl and/or hydroxyl groups.

21. The patch of claim 19 further comprising cationic metals for forming ionic bonds between patch and tissue surfaces.

22. The patch of claim 19, wherein the moieties are side chains of the biocompatible polymeric material.

23. The patch of claim 20, wherein the biocompatible polymeric material comprises a hydrogel including gelatin methacroyl modified with acrylic acid.

24. The patch of claim 1, wherein the auxetic architecture has maximum stiffness ratio of 1 to 5 and a maximum Poisson's ration of −1.2 to −0.05.

25-57. (canceled)

58. The patch of claim 3, wherein the hydrogel is selected from the group consisting of GelMA, PEGDA, gelatin norbornene, methacrylated decellularized ECM, and hyaluronic acid methacrylate.

59. The patch of claim 4, wherein the multiple hydrogel layers are selected from the group consisting of GelMA, PEGDA, gelatin norbornene, methacrylated decellularized ECM, and hyaluronic acid methacrylate.

60. The patch of claim of claim 5, wherein the differing hydrogel materials are selected from the group consisting of GelMA, PEGDA, gelatin norbornene, methacrylated decellularized ECM, and hyaluronic acid methacrylate.
